(19) United States
(12) Patent Application Publication
(10) Pub. No.: US 2026/0367097 A1
(43) Pub. Date: Nov. 19, 2026

(54) CALIBRATION INDEPENDENT IMMUNOASSAY SYSTEMS AND METHODS FOR COMPARATIVE ANALYTE DETECTION

(71) Applicant: Beckman Coulter, Inc., Brea, CA (US)

(72) Inventors: Miklos SZABO, Chaska, MN (US); Dusten UNRUH, Eden Prairie, MN (US); Brian Engel, Minneapolis, MN (US)

(21) Appl. No.: 19/200,491

(22) Filed: May 6, 2025

Related U.S. Application Data
(60) Provisional application No. 63/642,953, filed on May 6, 2024.
(60) Provisional application No. 63/690,217, filed on Sep. 3, 2024.
(60) Provisional application No. 63/725,169, filed on Nov. 26, 2024.
(60) Provisional application No. 63/774,434, filed on Mar. 19, 2025.

(51) Int. Cl. G01N 33/68; G01N 21/17; G01N 21/64

(52) U.S. Cl. CPC G01N 33/6896; G01N 21/17; G01N 21/6428; G01N 2021/1738; G01N 2333/775; G01N 2800/2821; G01N 2800/50

(57) ABSTRACT

The presently described and claimed technology relates to high-throughput automated methods for the investigation of ApoE isoforms the subsequent prediction of a likelihood of developing a neurodegenerative disease.

### CROSS-REFERENCE TO RELATED APPLICATIONS

This patent application claims priority to International Patent Application Nos. PCT/US25/27933, filed May 6, 2025, and PCT/US25/27937, filed May 6, 2025, U.S. Provisional Application No. 63/642,953, filed on May 6, 2024, 63/690,217, filed Sep. 3, 2024, 63/725,169, filed Nov. 26, 2024, and 63/774,434, filed Mar. 19, 2025, each of which is hereby incorporated by reference herein.

### INCORPORATION BY REFERENCE OF MATERIAL SUBMITTED ELECTRONICALLY

The entire content of a Sequence Listing titled 67811_Sequence_Listings.xml, created on Sep. 10, 2025, and having a size of 4,657 bytes, which is being submitted electronically in connection with the present application, is incorporated by reference herein in its entirety.

### BACKGROUND

Dementia is one of the costliest conditions to society. In 2022, the total national cost of caring for people living with Alzheimer's Disease (AD) and other dementias was projected to reach $321 billion, and an estimated 6.5 million Americans aged 65 and older were estimated to be living with AD. Am J Manag Care. 2022; 28 (suppl 10):S188-S196. By 2060, the number of people aged 65 and older with AD is projected to reach 13.8 million, barring the development of medical breakthroughs to prevent, slow or cure AD. Alzheimer's Association. 2022 Alzheimer's Disease Facts and Figures. Alzheimers Dement 2022; 18. Dementia and AD are costly to society because affected individuals often require long-term care and support, and available treatments aim to manage symptoms and delay progression, rather than to reverse the underlying disease process. Accordingly, AD is impacting a growing population that is currently underserved with treatment options. Novel treatments for AD are emerging and driving an increased need for testing and diagnosis.

Amyloid-Related Imaging Abnormalities (ARIA) are adverse events often reported in clinical trials for disease-modifying therapies (DMTs), especially for AD treatments involving amyloid-beta (AB) targeting therapies. ARIA can manifest as ARIA-E (edema or swelling) or ARIA-H (hemorrhage or bleeding), both detectable on MRI scans. Due to the risks associated with ARIA, monitoring for these abnormalities is crucial in clinical trials and later in clinical practice when administering these DMTs. Effective triaging for DMT eligibility requires identifying patients' risk factors for ARIA, such as age, genetic predispositions (e.g., APOE ε4 status), and baseline MRI findings, to ensure the safe and effective delivery of these therapies.

Assays are an important analytical tool for the identification and detection of specific substances in a sample and are routinely used to detect and quantify clinically important blood proteins. Many medical decisions are based on the diagnostic results from these assays, making sensitivity and specificity extremely important. Automated analyzers are commonly used in clinical chemistry, immunoassay, hematology, and other biological sampling and analyzing assays and can efficiently perform clinical analysis on a large number of samples, with multiple tests being run concurrently or within short time intervals.

APOE4 (apolipoprotein E4) zygosity is a well-established genetic predictor of AD risk. Individuals with one copy of the APOE4 allele (heterozygous) have an elevated risk of developing AD, while those with two copies (homozygous) have an even higher risk and often experience earlier onset. APOE4 influences AD risk by affecting amyloid-beta accumulation and neuroinflammation, contributing to the pathology of AD. This genetic marker is used in research and clinical settings to assess AD risk and inform decisions on preventive or therapeutic approaches.

However, the APOE4 genotype, especially in individuals with one or two copies of the ε4 allele, is linked to a heightened risk of ARIA when undergoing certain Alzheimer's disease (AD) treatments, particularly those targeting amyloid-beta. Due to this increased risk, APOE4 carriers may require more intensive monitoring, particularly during the first year of treatment, to detect and manage ARIA events early.

A need exists for a combined ApoE assay based diagnostic method that is sensitive and specific enough to predict the likelihood of developing a neurodegenerative disease.

### BRIEF SUMMARY

One aspect of the disclosure includes a method of assessing a subject's likelihood of developing a neurodegenerative disease, the method comprising contacting a first portion of at least one biological sample from the subject with first and second apolipoprotein (ApoE) antibodies, wherein the second ApoE antibody is specific to a single apolipoprotein E isoform, generating a first reaction mixture; conducting a detection reaction in the first reaction mixture generating a first detection signal; detecting the first detection signal; contacting a second portion of the at least one biological sample from the subject with third and fourth ApoE antibodies capable of binding to multiple ApoE isoforms, wherein the third or fourth ApoE antibody and the first ApoE antibody bind to a same epitope of ApoE, generating a second reaction mixture; conducting a detection reaction in the second reaction mixture generating a second detection signal; detecting the second detection signal; calculating a ratio of the first detection signal to the second detection signal; assigning the subject a degree of likelihood that the subject will develop a neurodegenerative disease and/or an accuracy of diagnosis of a neurodegenerative disease based on comparison of the ratio to a ratio established in a reference population.

One aspect of the disclosure includes method of assessing a subject's likelihood of developing a neurodegenerative disease, the method comprising: exposing a first portion of a biological sample from the subject to a first capture antibody capable of binding to Apolipoprotein E (ApoE) and a first detector affinity molecule specific to a single isoform of ApoE apolipoprotein, generating a first reaction mixture; conducting a detection reaction in the first reaction mixture wherein the first detector affinity molecule generates a first detection signal; recording the first detection signal; exposing a second portion of the at least one biological sample from the subject to a second capture antibody capable of binding to ApoE and a second detector affinity molecule capable of binding to multiple isoforms of ApoE, generating a second reaction mixture, wherein the first capture antibody and the second capture antibody bind to the same epitope of ApoE; conducting a detection reaction in the second reaction mixture wherein the second detector affinity molecule generates a second detection signal; calculating a ratio of the recorded first detection signal to the recorded second detection signal to generate an ApoE isomer comparison value and assigning, based on the comparison, a degree of likelihood that the subject will develop a neurodegenerative disease and/or an accuracy of diagnosis of a neurodegenerative disease.

One aspect of the disclosure is a method of assessing a degree of likelihood that a subject will be at risk of developing ARIA, the method comprising contacting a first portion of at least one biological sample from the subject with first and second apolipoprotein (ApoE) antibodies, wherein the second ApoE antibody is specific to apolipoprotein E4 isoform (ApoE4), generating a first reaction mixture; conducting a detection reaction in the first reaction mixture generating a first signal; detecting the first signal; contacting a second portion of the at least one biological sample from the subject with third and fourth ApoE antibodies capable of binding to multiple ApoE isoforms, wherein the third or fourth ApoE antibody and the first ApoE antibody bind to a same epitope of ApoE, generating a second reaction mixture; conducting a detection reaction in the second reaction mixture generating a second signal; detecting the second signal; calculating a ratio of the first signal to the second signal; assigning the subject an APOE ε4 genotype based on comparison of the ratio to a ratio established in a reference population, and assessing a degree of likelihood that the subject will be at risk of developing ARIA based on the whether the subject is heterozygous or homozygous for the APOE ε4 allele.

One aspect of the disclosure is A method of assessing a degree of likelihood that a subject will be at risk of developing ARIA, the method comprising exposing a first portion of at least one biological sample from the subject to a first capture antibody capable of binding to Apolipoprotein E (ApoE) and a first detector affinity molecule specific to apolipoprotein E4 isoform (ApoE4), generating a first reaction mixture; conducting a detection reaction in the first reaction mixture wherein the first detector affinity molecule generates a first detection signal; recording the first detection signal; exposing a second portion of the at least one biological sample from the subject to a second capture antibody capable of binding to ApoE and a second detector affinity molecule capable of binding to multiple isoforms of ApoE, generating a second reaction mixture, wherein the first capture antibody and the second capture antibody bind to the same epitope of ApoE; conducting a detection reaction in the second reaction mixture wherein the second detector affinity molecule generates a second detection signal; recording the second detection signal; calculating a ratio of the recorded first detection signal to the recorded second detection signal to generate an ApoE4 comparison value and, predicting, based on the ApoE4 comparison value, whether the subject is heterozygous or homozygous for the APOE ε4 allele and assessing a degree of likelihood that the subject will be at risk of developing ARIA based on the whether the subject is heterozygous or homozygous for the APOE ε4 allele.

In an aspect, the single isoform is ApoE4. In an aspect, the single isoform is ApoE2. In an aspect, the single isoform is ApoE3.

In an aspect, the first capture antibody and the second capture antibody are the same.

In an aspect, the subject has mild cognitive impairment and/or is 55 years or older.

In an aspect, the degree of likelihood that the subject is at risk of developing a neurodegenerative disease is low risk, medium risk, or high risk.

In an aspect, the neurodegenerative disease is Alzheimer's disease.

In an aspect, if the first detection signal is equal to or less than a reference signal established in a low risk reference population, the subject is assigned a low risk degree of likelihood.

In an aspect, if the first detection signal is higher than a reference signal established in a low risk reference population, the subject is assigned a medium or high risk degree of likelihood.

In an aspect, if the ratio is about equal to a certain reference ratio range established in a high risk reference population, the subject is assigned a high risk degree of likelihood.

In an aspect, if the ratio is at least about 0.5×s lower than a reference signal ratio established in a high risk reference population, the subject is assigned a medium risk degree of likelihood.

In an aspect, the accuracy of diagnosis results in less false positive and/or less false negative assignments.

In an aspect, the method further comprises assigning the subject an APOE ε4 genotype based on the comparison of the ratio of ApoE isomer comparison value to a ratio or value established in a reference population.

In an aspect, the method further comprises assessing a degree of likelihood that the subject will be at risk of developing Amyloid-Related Imaging Abnormalities (ARIA) based on the whether the subject is heterozygous or homozygous for the APOE ε4 allele.

In an aspect, the degree of likelihood that the subject will develop ARIA is low risk, medium risk, or high risk.

In an aspect, the subject is recommend further treatment.

In an aspect, the further treatment comprises qualifying the subject for a clinical trial, assessing the risks associated with the subject entering the clinical trial, and/or increasing safeguards for the subject in clinical trial.

In an aspect, the further treatment comprises assessing a subject's eligibility for a disease-modifying therapy (DMT), administering a DMT and/or providing monitoring or additional treatments in conjunction with the DMT.

In an aspect, the DMT is selected from the group consisting of anti-amyloid therapies, anti-tau therapies, immunotherapies, vaccines, BACE inhibitors, anti-inflammatory drugs, neurotrophic drugs, and combinations thereof.

In an aspect, the detector affinity molecule is an antibody, a monoclonal antibody, a polyclonal antibody, an antibody fragment, a synthetic antibody mimic, an aptamer, an affimer, DARPins, oligonucleotide, peptide, or antigen.

In an aspect, the capture antibody, the first detector affinity molecule, and/or the second detector affinity molecule is conjugated to at least one magnetic bead.

In an aspect, the first detector affinity molecule and/or the second detector affinity molecule is conjugated to an enzyme.

In an aspect, the enzyme comprises horseradish peroxidase or alkaline phosphatase.

In an aspect, the method is performed using an immunoassay analyzer, wherein the immunoassay analyzer comprises: a pipettor arrangement comprising at least one reagent pipettor and at least one sample pipettor; and a detector arrangement.

In an aspect, the detector arrangement comprises a luminometer, an electrochemiluminescence (ECL) detector, a photomultiplier tube (PMT) detector, a photometer, a fluorometer, or a bioluminescence detector.

In an aspect, the first detection signal and/or the second detection signal are luminescent signals, eletrochemiluminescent signals, or chemiluminescent signals.

In an aspect, the first detection signal and/or the second detection signal are generated via a detection reaction between the first detector affinity molecule and/or the second detector affinity molecule and a substrate formulation, wherein the substrate formulation comprises: a chemiluminescent compound of the formula A or a salt thereof:

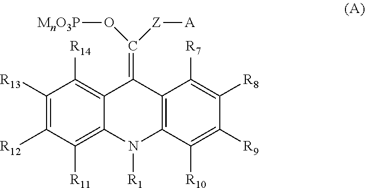

wherein A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁₋₆ alkyl, C₁₋₆ haloalkyl, and C₅₋₁₄ aralkyl groups;
R₇-R₁₄ are independently H, C₁₋₆ alkoxy, halo, C₁₋₄ alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2;
a cationic aromatic compound (CAC);
a background reducing agent; and
an ether-linked nonionic surfactant or a hydrophilic polymer.

In an aspect, immunoassay analyzer further comprises at least one reagent pack configured to hold a plurality of reagent vessels, wherein each reagent vessel is configured to store a volume of reagent required for at least one instance of the assays. In an aspect, the reagent pack comprises a reagent vessel comprising the first capture antibody, a reagent vessel comprising the first detector affinity molecule, and a reagent vessel comprising the second detector affinity molecule.

In an aspect, the immunoassay analyzer further comprises an ultrasonic mixing module.

In an aspect, the immunoassay analyzer further comprises a machine vision apparatus comprising an image capture device and an image interpretation device configured to monitor instrument and/or assay functionalities of the immunoassay analyzer.

In an aspect, the instrument functionalities are selected from the group consisting of optical sensors, pressure sensors and thermistors.

In an aspect, the assay functionalities are selected from the group consisting of sample volume monitoring, total reagent volume monitoring, residual volume monitoring, and particle retention monitoring.

In an aspect, the pipettor arrangement comprises at least a first reagent pipettor, a second reagent pipettor, and a third reagent pipettor.

In an aspect, the pipettor arrangement further comprises at least a fourth reagent pipettor.

In an aspect, the first reagent pipettor, second reagent pipettor, third reagent pipettor, and/or fourth reagent pipettor is selectively and/or simultaneously operated.

In an aspect, the first reagent pipettor, second reagent pipettor, third reagent pipettor, and/or fourth reagent pipettor are configured to engage a dispense tip prior to aspiration.

In an aspect, the first reaction mixture, the second reaction mixture, and/or the third reaction mixture comprise unreacted components, and the immunoassay analyzer further comprises a washing arrangement, wherein the washing arrangement is configured to perform at least one wash action to wash away at least a portion of unreacted components, alternatively configured to perform at least two wash actions, alternatively configured to perform at least three wash actions, alternatively configured to perform at least four wash actions, alternatively configured to perform at least five wash actions, alternatively configured to perform at least six wash actions, alternatively configured to perform at least seven wash actions, alternatively configured to perform at least eight wash actions, alternatively configured to perform at least nine wash actions, or alternatively configured to perform at least ten wash actions.

In an aspect, the first reaction mixture, the second reaction mixture, and/or the third reaction mixture is subjected to a magnetic field prior to performing the at least one wash action.

In an aspect, the biological sample is serum, whole blood, plasma, and/or cerebral spinal fluid. In an aspect, the biological sample volume is less than about 10 μL, alternatively between about 2 μL to about 9.9 μL. In an aspect, the method is configured to analyze at least about 200 biological samples/hr; alternatively at least about 300 biological samples/hr; or alternatively at least about 400 biological samples/hr.

Another aspect of the disclosure is a system for assessing a relative presence of a first analyte subgroup of a total analyte in a sample, wherein the total analyte present in the sample includes the first analyte subgroup and at least one additional analyte subgroup, the system comprising: an immunoassay analyzer comprising a pipettor arrangement; an analytic unit; a detector arrangement; and a non-transitory computer-readable medium storing instructions that, when executed by a processor, cause the system to perform a series of steps comprising: a first step comprising assaying the sample to produce a first quantitative signal corresponding to the first analyte subgroup; a second step comprising assaying the sample to produce a second quantitative signal corresponding to the total analyte or the at least one additional analyte subgroup; and a third step comprising determining the relative presence of the first analyte subgroup of the total analyte in the sample by relatively comparing the two quantitative signals without reference to a calibration curve, wherein either all subgroups of the total analyte in the sample share a common epitope, or the first analyte subgroup and the at least one additional analyte subgroup share a common epitope; and wherein the assaying the sample steps to produce the first and second quantitative signals further comprise binding a capture agent to the common epitope.

In an aspect, the first step of assaying the sample to produce the first quantitative signal and the second step of assaying the sample to produce the second quantitative signal are not performed concurrently. In an aspect, the second step comprising assaying the sample to produce a second quantitative signal corresponds to the total analyte present in the sample. In some embodiments, the first step of assaying the sample to produce the first quantitative signal further comprises exposing the sample to a first detection agent specific to the first analyte subgroup in the sample and the second step of assaying the sample to produce the second quantitative signal further comprises exposing the sample to a second detection agent capable of binding to the total analyte in the sample. In some embodiments, the first step of assaying the sample to produce the first quantitative signal further comprises exposing the sample to a first detection agent specific to the first analyte subgroup in the sample and the second step of assaying the sample to produce the second quantitative signal further comprises exposing the sample to a second detection agent specific to the at least one additional analyte subgroup in the sample. In some embodiments, the first and second quantitative signals are generated based on bulk signal accumulation and not individual binding events. In some embodiments, the first and second steps comprising assaying the sample comprise the use of magnetic particle-based immunoassays performed in two separate reaction vessels. In some embodiments, the immunoassays performed are subject to differing reaction conditions. In some embodiments, the differing reaction conditions comprise different incubation times, mix times, or reagent additions. In some embodiments, the immunoassay comprises a chemiluminiscence immunoassay (CLIA), immunofluorescence assay, fluorescence polarization assay, one-site noncompetitive assay, or two-site noncompetitive (sandwich) immunoassay. In some embodiments, the detector arrangement comprises a luminometer, an electrochemiluminescence (ECL) detector, a photomultiplier tube (PMT) detector, a photometer, a fluorometer, or a bioluminescence detector. In some embodiments, the two quantitative signals are luminescent signals, eletrochemiluminescent signals, or chemiluminescent signals.

In an aspect, the analyte comprises a peptide, a polypeptide, an enzyme, a protein, a protein complex, a monoclonal antibody, a polyclonal antibody, an antibody fragment, a hormone, a synthetic antibody mimic, an aptamer, an affimer, DARPins, oligonucleotide, a nucleic acid, or an antigen. In some embodiments, the first analyte subgroup is structurally or chemically different from the at least one additional analyte subgroup. In some embodiments, the sample comprises a biological sample from a patient, wherein the biological sample is serum, whole blood, plasma, cerebral spinal fluid, urine, feces, sputum, tissue, marrow, semen, mucus, or nasopharyngeal fluid.

In an aspect, the first step comprising assaying the sample to produce a first quantitative signal corresponding to the first analyte subgroup comprises: contacting a first portion the sample with a capture antibody specific for the analyte to produce a first capture mixture; contacting the first capture mixture with a detector affinity molecule that is specific for the first analyte subgroup to produce a first detection mixture; conducting a first detection reaction in the first detection mixture generating a first quantitative signal; and detecting the first quantitative signal; and the second step comprising assaying the sample to produce a second quantitative signal corresponding to the total presence of analyte or the at least one additional analyte subgroup comprises: contacting a second portion the sample with a capture antibody specific for the analyte to produce a second capture mixture; contacting the second capture mixture with a detector affinity molecule that is specific for the analyte or the at least one additional analyte to produce a second detection mixture; conducting a second detection reaction in the second detection mixture generating a second quantitative signal; anddetecting the second quantitative signal.

In an aspect, the detector affinity molecule is an antibody, a monoclonal antibody, a polyclonal antibody, an antibody fragment, a synthetic antibody mimic, an aptamer, an affimer, DARPins, oligonucleotide, peptide, or antigen. In some embodiments, the first capture antibody and/or the second capture antibody is coupled to a particle or bead that is optionally magnetic.

In an aspect, the first detector affinity molecule, and/or the second detector affinity molecule is conjugated to at least one enzyme. In some embodiments, the enzyme comprises horseradish peroxidase or alkaline phosphatase.

In an aspect, the two quantitative signals are generated via a detection reaction between the first detector affinity molecule and/or the second detector affinity molecule and a substrate formulation, wherein the substrate formulation comprises: a chemiluminescent compound of the formula A or a salt thereof:

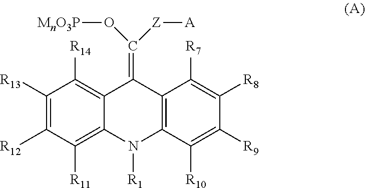

Wherein A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents; R₁ is selected from the group consisting of C₅₋₁₄aryl, C₁₋₆ alkyl, C₁₋₆ haloalkyl, and C₅₋₁₄ aralkyl groups; R₇-R₁₄ are independently H, C₁₋₆ alkoxy, halo, C₁₋₄ alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring; R₁₅ is C₁₋₆ alkyl; each M is independently selected from the group consisting of H, an alkali metal, alkaline earth metal, transition metal, ammonium, phosphonium, organic amine salt, and an amino acid salt; Z is O or S; and n is 0, 1, or 2; a cationic aromatic compound (CAC); a background reducing agent; and an ether-linked nonionic surfactant or a hydrophilic polymer.

In an aspect, the immunoassay analyzer further comprises at least one reagent pack configured to hold a plurality of reagent vessels, wherein each reagent vessel is configured to store a volume of at least one reagent required for at least one instance of the assays. In some embodiments, the reagent pack comprises a reagent vessel comprising the first and second capture antibodies, a reagent vessel comprising the first detector affinity molecule, and a reagent vessel comprising the second detector affinity molecule.

In an aspect, the the immunoassay analyzer further comprises one or more of an ultrasonic mixing module or a machine vision apparatus comprising an image capture device and an image interpretation device configured to monitor instrument and/or assay functionalities of the immunoassay analyzer, wherein the instrument functionalities are optionally selected from the group consisting of optical sensors, pressure sensors and thermistors and the assay functionalities are optionally selected from the group consisting of sample volume monitoring, total reagent volume monitoring, residual volume monitoring, and particle retention monitoring.

In an aspect, the pipettor arrangement comprises at least a first reagent pipettor, a second reagent pipettor, and a third reagent pipettor. In some embodiments, the pipettor arrangement further comprises at least a fourth reagent pipettor. In some embodiments, the first reagent pipettor, second reagent pipettor, third reagent pipettor, and/or fourth reagent pipettor is selectively and/or simultaneously operated.

In an aspect, the first step comprising assaying the sample is done in a first reaction vessel to produce a first reaction mixture comprising unreacted components, and the immunoassay analyzer further comprises a washing arrangement, wherein the washing arrangement is configured to perform at least one wash action to wash away at least a portion of unreacted components; and the second step comprising assaying the sample is done in a second reaction vessel to produce a second reaction mixture comprising unreacted components, and the immunoassay analyzer further comprises a washing arrangement, wherein the washing arrangement is configured to perform at least one wash action to wash away at least a portion of unreacted components. In some embodiments, the first reaction mixture and/or the second reaction mixture is subjected to a magnetic field prior to performing the at least one wash action.

In an aspect, the immunoassay analyzer is configured to analyze at least about 200 biological samples per hour.

Another aspect comprises a method of assessing a relative presence of a first analyte subgroup of a total analyte in a sample, wherein the total analyte present in the sample includes the first analyte subgroup and at least one additional analyte subgroup, the method comprising: assaying the sample to produce a first quantitative signal corresponding to the first analyte subgroup; assaying the sample to produce a second quantitative signal corresponding to the total analyte or the at least one additional analyte subgroup; and determining, based on a relative comparison of the two quantitative signals, and without reference to a calibration curve, the relative presence of the first analyte subgroup, wherein either all subgroups of the total analyte share a common epitope, or the first analyte subgroup and the at least one additional analyte subgroup share a common epitope; and wherein the assaying the sample to produce the first and second quantitative signals comprises binding a capture agent to the common epitope. In some embodiments, the assaying the sample steps to produce the first and second quantitative signals are not performed concurrently. In some embodiments, the method comprises assaying the sample to produce a second quantitative signal corresponding to the total analyte present. In some embodiments, the assaying the sample to produce the first and second quantitative signals further comprises exposing the sample to a first detection agent specific to the first analyte subgroup and a second detection agent capable of binding to the total analyte. In some embodiments, the assaying the sample to produce the first and second quantitative signals further comprises exposing the sample to a first detection agent specific to the first analyte subgroup and a second detection agent specific to the at least one additional analyte subgroup. In some embodiments, the first and second quantitative signals are generated based on bulk signal accumulation and not individual binding events.

In an aspect, the assaying comprises performing one or more immunoassays. In some embodiments, the one or more immunoassays are magnetic particle-based immunoassays performed in two separate reaction vessels. In some embodiments, the one or more immunoassays performed are subject to differing reaction conditions. In some embodiments, the differing reaction conditions comprise different incubation times, mix times, or reagent additions.

In an aspect, the analyte comprises a peptide, a polypeptide, an enzyme, a protein, a protein complex, a monoclonal antibody, a polyclonal antibody, an antibody fragment, a hormone, a synthetic antibody mimic, an aptamer, an affimer, DARPins, oligonucleotide, a nucleic acid, or an antigen. In some embodiments, the first analyte subgroup is structurally or chemically different from the at least one additional analyte subgroup.

In an aspect, the sample comprises a biological sample from a patient, wherein the biological sample is serum, whole blood, plasma, cerebral spinal fluid, urine, feces, sputum, tissue, marrow, semen, mucus, or nasopharyngeal fluid. In some embodiments, the sample is a plasma sample.

In an aspect, the immunoassay comprises a Western Blot, immunoprecipitation, radioimmunoassay, enzyme (ELISA), chemiluminiscence immunoassay (CLIA), immunofluorescence, immunohistochemistry, immunocytochemistry, flow cytometry, fluorescence polarization, enzyme multiplied, luminescent oxygen channeling, particle enhanced turbidimetric inhibition, cloned enzyme donor, one-site noncompetitive, or two-site noncompetitive (sandwich) immunoassays. In some embodiments, the immunoassay is performed on an automated immunoassay analyzer. In some embodiments, the assaying of the at least two quantitative signals is performed simultaneously.

In an aspect, assaying the sample to produce a first quantitative signal corresponding to the first analyte subgroup comprises: contacting a first portion the sample with a capture antibody specific for the analyte to produce a first capture mixture; contacting the first capture mixture with a capture antibody specific for the analyte to produce a first detection mixture; conducting a first detection reaction in the first detection mixture generating a first quantitative signal; and detecting the first quantitative signal; and assaying the sample to produce a second quantitative signal corresponding to the total presence of analyte or the at least one additional analyte subgroup comprises: contacting a second portion the sample with a capture antibody specific for the analyte to produce a second capture mixture; contacting the second capture mixture with a detector affinity molecule that is specific for the analyte or the at least one additional analyte to produce a second detection mixture; conducting a second detection reaction in the second detection mixture generating a second quantitative signal; and detecting the second quantitative signal.

In an aspect, the detector affinity molecule is an antibody, a monoclonal antibody, a polyclonal antibody, an antibody fragment, a synthetic antibody mimic, an aptamer, an affimer, DARPins, oligonucleotide, peptide, or antigen. In some embodiments, the first capture antibody and/or the second capture antibody is coupled to at least one magnetic bead. In some embodiments, the first detector affinity molecule, and/or the second detector affinity molecule is conjugated to at least one enzyme. In some embodiments, the enzyme comprises horseradish peroxidase or alkaline phosphatase.

In an aspect, the assaying is performed using an immunoassay analyzer, wherein the immunoassay analyzer comprises: a pipettor arrangement comprising at least one reagent pipettor and at least one sample pipettor; and a detector arrangement. In some embodiments, the detector arrangement comprises a luminometer, an electrochemiluminescence (ECL) detector, a photomultiplier tube (PMT) detector, a photometer, a fluorometer, or a bioluminescence detector. In some embodiments, the at least two quantitative signals are luminescent signals, eletrochemiluminescent signals, or chemiluminescent signals.

In an aspect, the two quantitative signals are generated via a detection reaction between the first detector affinity molecule and/or the second detector affinity molecule and a substrate formulation, wherein the substrate formulation comprises: a chemiluminescent compound of the formula A or a salt thereof:

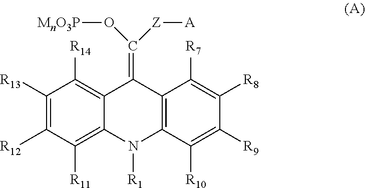

Wherein A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents; R₁ is selected from the group consisting of C₅₋₁₄aryl, C₁₋₆ alkyl, C₁₋₆ haloalkyl, and C₅₋₁₄ aralkyl groups; R₇-R₁₄ are independently H, C₁₋₆ alkoxy, halo, C₁₋₄ alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring; R₁₅ is C₁₋₆ alkyl; each M is independently selected from the group consisting of H, an alkali metal, alkaline earth metal, transition metal, ammonium, phosphonium, organic amine salt, and an amino acid salt; Z is O or S; and n is 0, 1, or 2; a cationic aromatic compound (CAC); a background reducing agent; and an ether-linked nonionic surfactant or a hydrophilic polymer.

In an aspect, the immunoassay analyzer further comprises at least one reagent pack configured to hold a plurality of reagent vessels, wherein each reagent vessel is configured to store a volume of at least one reagent required for at least one instance of the assays. In some embodiments, the reagent pack comprises a reagent vessel comprising the first and second capture antibodies, a reagent vessel comprising the first detector affinity molecule, and a reagent vessel comprising the second detector affinity molecule.

In an aspect, the the immunoassay analyzer further comprises one or more of an ultrasonic mixing module or a machine vision apparatus comprising an image capture device and an image interpretation device configured to monitor instrument and/or assay functionalities of the immunoassay analyzer, wherein the instrument functionalities are optionally selected from the group consisting of optical sensors, pressure sensors and thermistors and the assay functionalities are optionally selected from the group consisting of sample volume monitoring, total reagent volume monitoring, residual volume monitoring, and particle retention monitoring.

In an aspect, the pipettor arrangement comprises at least a first reagent pipettor, a second reagent pipettor, and a third reagent pipettor. In some embodiments, the pipettor arrangement further comprises at least a fourth reagent pipettor. In some embodiments, the first reagent pipettor, second reagent pipettor, third reagent pipettor, and/or fourth reagent pipettor is selectively and/or simultaneously operated.

In an aspect, the first step comprising assaying the sample is done in a first reaction vessel to produce a first reaction mixture comprising unreacted components, and the immunoassay analyzer further comprises a washing arrangement, wherein the washing arrangement is configured to perform at least one wash action to wash away at least a portion of unreacted components; and the second step comprising assaying the sample is done in a second reaction vessel to produce a second reaction mixture comprising unreacted components, and the immunoassay analyzer further comprises a washing arrangement, wherein the washing arrangement is configured to perform at least one wash action to wash away at least a portion of unreacted components. In some embodiments, the first reaction mixture and/or the second reaction mixture is subjected to a magnetic field prior to performing the at least one wash action.

In an aspect, the immunoassay analyzer is configured to analyze at least about 200 biological samples per hour.

Another aspect of the method comprises assigning a genotype, assessing a biomedical diagnostic value, diagnosing a condition, staging a disease, determining a prognosis, detecting a medical deviation, or determining a treatment in the subject based on the relative presence of the first analyte subgroup compared to the total presence of analyte or the at least one additional analyte subgroup

In some aspects, (a) the first analyte subgroup and (b) the at least one additional analyte subgroup or at least one molecule comprising the total analyte share a common epitope and differ by or post-translational modification and/or a proteolytic cleavage event.

Another aspect of the method comprises assaying the sample to produce a second quantitative signal corresponding to the at least one additional analyte subgroup, wherein the first analyte subgroup comprises a full-length form of a protein and the at least one additional analyte subgroup comprises a truncated form of the protein, a phosphorylated form of the protein, and/or a non-phosphorylated form of the protein. In some embodiments, the first analyte subgroup comprises a full-length phosphorylated form of the protein and the at least one additional analyte subgroup comprises a full-length non-phosphorylated form of the protein.

Another aspect of the method comprises assaying the sample to produce a second quantitative signal corresponding to the total analyte, wherein the analyte comprises a protein and wherein the first analyte subgroup comprises a full-length form of the protein, a truncated form of the protein, a phosphorylated form of the protein, and/or a non-phosphorylated form of the protein. In some embodiments, the first analyte subgroup comprises a tau protein phosphorylated at a residue selected from Thr181, Ser198, Ser199, Ser202, Thr217, Thr231, Ser262, Ser396, or Ser422; the total analyte comprises all tau proteins, and the at least one additional analyte subgroup comprises a tau protein unphosphorylated at a residue, wherein the unphosphorylated residue is the same residue as in the first analyte subgroup. In some embodiments, the residue is Thr181, Thr217, or Thr231.

In another aspect of the method, the first analyte subgroup and the at least one additional analyte subgroup correspond to protein isoforms encoded by different alleles of a gene. In some embodiments, the comparison of the first and second signals is indicative of a subject's genotype. In some embodiments, the comparison of the first and second signals is indicative of a subject's genotype. In some embodiments, the analyte comprises a haptoglobin isoform selected from genotypes Hp1-1, Hp2-1, and Hp2-2.

In another aspect of the method, the first analyte subgroup and the at least one additional analyte subgroup comprises a normal protein and a mutant or aberrant form of the protein resulting from a single gene disorder. In some embodiments, the first analyte subgroup and the at least one additional analyte subgroup comprises protein isoforms encoded by allelic variants of a gene that encodes a drug-metabolizing enzyme. In some embodiments, the first analyte and at least one additional analyte subgroup comprises protein isoforms with differing enzymatic activity resulting from underlying genetic polymorphism.

Another aspect comprises a kit for assessing a relative presence of a first analyte subgroup of a total analyte in a sample, wherein the total analyte present in the sample includes the first analyte subgroup and at least one additional analyte subgroup, the kit comprising: a first assay reagent set configured to measure a first quantitative signal corresponding to the first analyte subgroup in the sample; and a second assay reagent set configured to measure a second quantitative signal corresponding to the total analyte or the at least one additional analyte subgroup in the sample. In some embodiments, the first and second reagent sets are each configured for use on an immunoassay analyzer. In some embodiments, the first reagent set is configured for use on an immunoassay analyzer and the second reagent set is configured for use on a clinical chemistry analzyer. In some embodiments, the first assay reagent set comprises: a capture antibody specific for the analyte; a detector antibody specific for the first analyte subgroup; and the second assay reagent set comprises: a capture antibody specific for the analyte; and a detector affinity molecule that is specific for the analyte or the at least one additional analyte subgroup to produce a second detection mixture. In some embodiments, each detector affinity molecule is an antibody, a monoclonal antibody, a polyclonal antibody, an antibody fragment, a synthetic antibody mimic, an aptamer, an affimer, DARPins, oligonucleotide, peptide, or antigen. In some embodiments, the first capture antibody and/or the second capture antibody is coupled to at least one magnetic bead. In some embodiments, the first detector affinity molecule, and/or the second detector affinity molecule is conjugated to at least one enzyme.

Another aspect is a non-transitory machine-readable medium storing data configured for execution by an immunoassay analyzer, the data comprising instructions that cause the analyzer to perform the method of any of aspects or embodiments described herein.

These and other advantages, aspects, and novel features of the present disclosure, as well as details of illustrated embodiments thereof, will be more fully understood from the following description and drawings.

### BRIEF DESCRIPTION OF THE FIGURES

Embodiments of the present disclosure will now be described, by way of example only, with reference to the attached Figures, wherein:

FIG. 1 is a graph showing the role of APOE genotypes in Alzheimer's Disease monitoring.

FIG. 2 is a reagent pack according to an exemplary aspect of the disclosure.

FIG. 3 is a table of patient samples analyzed using a method according to an aspect of this disclosure and which have been assigned an ApoE4 genotype.

FIG. 4 is a precision assessment of patient samples analyzed using a method according to an aspect of this disclosure.

FIG. 5 is a graph of patient samples analyzed using a method according to an aspect of this disclosure and stratified according to ApoE4 genotype.

FIG. 6 is a graph of patient samples analyzed using a method according to an aspect of this disclosure. The graph shows replicate-to-replicate, lot-to-lot, and platform variability of samples in K2 EDTA plasma. Calculated results show low variability across 84 sample concentrations of low (square), medium (triangle) and high (circle). The study shows agreement in results interpretation across reps, reagent lots and testing platform.

### DETAILED DESCRIPTION

### I. Introduction

AD is a progressive neurodegenerative disorder that affects millions of individuals. It is thought that individuals with AD have amyloid accumulation in the brain which damages the synaptic function and forms neurofibrillary tangles. Apolipoprotein E (ApoE), encoded by the APOE gene, has been shown to have an essential role in lipid metabolism. In the central nervous system, APOE is mainly produced by astrocytes and microglia, and transports cholesterol to neurons via APOE receptors.

The APOE gene has three major allelic variants, 82, 83, and 84, which encode ApoE2, ApoE3, and ApoE4 protein isoforms. The isoforms vary from one another by one or two amino acids at residues 112 and 158 in the APOE protein. ApoE2 contains 112/158 cys/cys; ApoE3 contains 112/158 cys/arg; and ApoE4 contains 112/158 arg/arg. There are also six different genotypes, APOE2/2, APOE2/3, APOE3/3, APOE2/4, APOE3/4, and APOE4/4. ApoE2 can refer to any full length form of ApoE2, a variant of ApoE2, a fragment of ApoE2, and post-translationally modified forms of ApoE2. ApoE3 can refer to any full length form of ApoE3, a variant of ApoE3, a fragment of ApoE3, and post-translationally modified forms of ApoE3. ApoE4 can refer to any full length form of ApoE4, a variant of ApoE4, a fragment of ApoE4, and post-translationally modified forms of ApoE4. “Total APOE” in the context of a biological sample refers to the total amount of all isoforms, or all major isoforms, of the APOE protein in the sample, including APOE2, APOE3, and APOE4. In some aspects, an antibody that binds to all isoforms, or all major isoforms, of ApoE is referred to as an anti-total-ApoE antibody, a total-ApoE antibody, an anti-PAN-ApoE antibody, or a PAN-ApoE antibody.

In the brain, ApoE regulates neuronal and synaptic functions. Genome-wide association studies have confirmed that the ε4 allele of APOE is the strongest genetic risk factor for both early-onset AD and late onset or sporadic AD. Risk of AD is thought to be increased in individuals with one copy of the ε4 allele (ε2/ε4; ε3/ε4) or two copies (ε4/ε4). ApoE4 has been associated with numerous cellular pathways that may influence AD disease progression, such as amyloid clearance, mitochondrial function, autophagy, and inflammation. It is thought that APOE ε4 is associated with increased risk of progression from MCI to AD type dementia and impaired memory performance and increased risk of memory decline in middle aged (40-59 years) and elderly (60-85 years) people with MCI.

The amino acid sequences of exemplary ApoE isoforms are listed in Table 1.

TABLE 1

| SEQ | | |
|---|---|---|
| ID | | Identi- |
| NO | Amino Acid Sequence | fier |
| 1 | KVEQAVETEPEPELRQQTEWQSGQRWELALGRFW | ApoE2 |
| | DYLRWVQTLSEQVQEELLSSQVTQELRALMDETMK | |
| | ELKAYKSELEEQLTPVAEETRARLSKELQAAQARLG | |
| | ADMEDVCGRLVQYRGEVQAMLGQSTEELRVRLAS | |
| | HLRKLRKRLLRDADDLQKCLAVYQAGAREGAERG | |
| | LSAIRERLGPLVEQGRVRAATVGSLAGQPLQERAQA | |
| | WGERLRARMEEMGSRTRDRLDEVKEQVAEVRAKL | |
| | EEQAQQIRLQAEAFQARLKSWFEPLVEDMQRQWA | |
| | GLVEKVQAAVGTSAAPVPSDNH | |
| 2 | KVEQAVETEPEPELRQQTEWQSGQRWELALGRFW | ApoE3 |
| | DYLRWVQTLSEQVQEELLSSQVTQELRALMDETMK | |
| | ELKAYKSELEEQLTPVAEETRARLSKELQAAQARLG | |
| | ADMEDVCGRLVQYRGEVQAMLGQSTEELRVRLAS | |
| | HLRKLRKRLLRDADDLQKRLAVYQAGAREGAERG | |
| | LSAIRERLGPLVEQGRVRAATVGSLAGQPLQERAQA | |
| | WGERLRARMEEMGSRTRDRLDEVKEQVAEVRAKL | |
| | EEQAQQIRLQAEAFQARLKSWFEPLVEDMQRQWA | |
| | GLVEKVQAAVGTSAAPVPSDNH | |
| 3 | KVEQAVETEPEPELRQQTEWQSGQRWELALGRFW | ApoE4 |
| | DYLRWVQTLSEQVQEELLSSQVTQELRALMDETMK | |
| | ELKAYKSELEEQLTPVAEETRARLSKELQAAQARLG | |
| | ADMEDVRGRLVQYRGEVQAMLGQSTEELRVRLAS | |
| | HLRKLRKRLLRDADDLQKRLAVYQAGAREGAERG | |
| | LSAIRERLGPLVEQGRVRAATVGSLAGQPLQERAQA | |
| | WGERLRARMEEMGSRTRDRLDEVKEQVAEVRAKL | |
| | EEQAQQIRLQAEAFQARLKSWFEPLVEDMQRQWA | |
| | GLVEKVQAAVGTSAAPVPSDNH | |

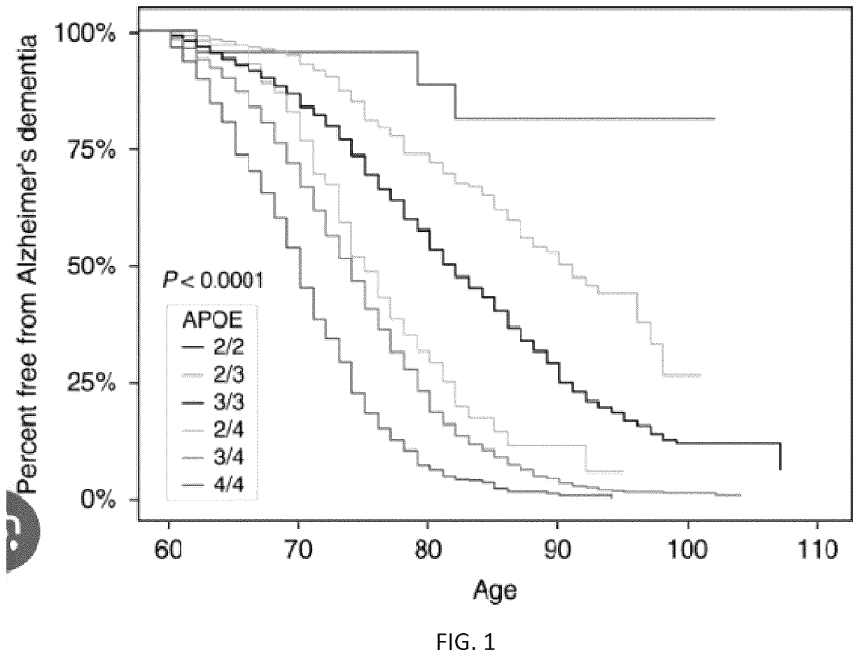

Several diagnostic tools are being developed to detect ApoE, in particular the ApoE4 isoform, to ascribe a person's risk or susceptibility for developing Alzheimer's disease (AD) or other neurological disorders. Without being limited by any theory, as shown in FIG. 1, individuals with APOE2/2, APOE2/3, and APOE3/3 genotypes have a low risk for developing AD. While individuals with APOE2/4 and APOE3/4 have a moderate risk (5 times higher) with and those with APOE4/4 having the highest risk (15 times higher).

Amyloid-Related Imaging Abnormalities (ARIA) refer to brain abnormalities that can appear as side effects in patients receiving certain disease-modifying therapies (DMTs) for Alzheimer's disease (AD), particularly those targeting amyloid-beta plaques. ARIA is typically categorized into two types: ARIA-E, which involves brain edema (swelling), and ARIA-H, which includes microhemorrhages (small brain bleeds) or superficial hemosiderosis (iron deposits from bleeding). These abnormalities are detectable by MRI and are believed to result from the inflammatory or vascular effects of amyloid-beta clearance, which may compromise blood-brain barrier integrity.

The APOE4 allele is a significant genetic factor influencing the risk of ARIA in patients undergoing amyloid-targeting therapies for AD. APOE4, especially in those carrying one (heterozygous) or two (homozygous) copies of the allele, is known to increase susceptibility to ARIA. Without being bound to any theory, this susceptibility may be due to the role of APOE4 in influencing blood-brain barrier integrity, amyloid clearance, and neuroinflammatory responses, all of which contribute to the development of these abnormalities. APOE4's impact on these pathways may exacerbate amyloid plaque-related vascular damage and inflammation, key mechanisms in ARIA formation during anti-amyloid treatment. In the context of AD treatments, especially with monoclonal antibodies designed to reduce amyloid-beta accumulation in the brain, ARIA is a critical adverse event to monitor due to its potentially serious effects. This association has made ARIA management a key focus in clinical trials and therapeutic protocols, where patient selection, genotyping, and rigorous MRI monitoring are used to balance treatment benefits with the risk of ARIA

Clinical trials and studies on amyloid-targeting drugs, such as certain monoclonal antibodies, have demonstrated that APOE4 carriers face a higher incidence of ARIA, particularly in the first months of treatment. Patients homozygous for APOE4 are at the highest risk, with some data suggesting as much as a threefold increase in ARIA risk compared to non-carriers. Consequently, genetic testing for APOE4 status is often considered in clinical settings to guide decisions on monitoring and to weigh the risks and benefits of treatment for each patient. In light of these findings, clinical trials for AD therapies have adapted their protocols to include APOE4 status as a consideration in eligibility and to determine monitoring intensity.

For APOE4 carriers undergoing anti-amyloid therapy, close monitoring with MRI is recommended to detect and manage ARIA events, particularly within the first year of treatment when the risk is highest. Regular imaging enables early identification of ARIA, allowing adjustments to treatment or temporary pauses if significant ARIA is detected. This monitoring approach aims to improve patient safety by reducing the likelihood of severe ARIA-related complications while still offering APOE4 carriers access to potentially beneficial therapies.

By incorporating APOE4 genotyping into trial protocols and clinical practice, clinicians can determine the need for additional precautions, such as more frequent MRI monitoring, dose adjustments, or alternative therapeutic approaches for high-risk individuals. These countermeasures are essential to mitigate ARIA-related complications and enhance the safety and efficacy of amyloid-targeting therapies for Alzheimer's disease.

High-throughput automated method for the investigation of ApoE isoforms, including the zygosity for the APOE ε4 allele, subsequent prediction of a likelihood of developing a neurodegenerative disease are disclosed herein.

### II. DEFINITIONS

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments or aspects does not imply that other embodiments or aspects are not useful and is not intended to exclude other embodiments or aspects from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” “one embodiment,” “an embodiment,” “certain embodiment,” or “some embodiment,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

As used herein, the terms “subject”, “individual”, and “patient” are interchangeable, and relate to vertebrates, preferably mammals. For example, mammals in the context of the disclosure are humans, non-human primates, domesticated animals such as dogs, cats, sheep, cattle, goats, pigs, horses, etc., laboratory animals such as mice, rats, rabbits, guinea pigs, etc., as well as animals in captivity such as animals in zoos. The term “animal” as used herein includes humans. The term “subject” may also include a patient, i.e., an animal, having a disease. In exemplary aspects, a subject, individual, or patient refers to a human (e.g., a man, a woman, or a child).

Automated analyzers are commonly used in clinical chemistry, immunoassay, hematology, and other biological sampling and analyzing applications. Automated analytical equipment, such as automated analytical chemistry instruments, automated analytical immunoassay instruments, automated analytical hematology instruments, etc., can efficiently perform clinical analysis on a large number of samples, with multiple tests being run concurrently or within short time intervals. Automated analytical instruments are particularly well-suited for high-volume and mid-volume testing environments. In some instances, methods disclosed herein are performed on an automated analyzer, such as a clinical chemistry analyzer or an immunoassay analyzer.

A “clinical chemistry analyzer” includes an automated analyzer that utilizes spectrometry, chromatography, photometry, or potentiometry principles to measure or calculate the concentration of certain substances. Some clinical chemistry analyzers apply the photoelectric colorimetric principle and employ at least one chemical reaction to measure or calculate the concentration of certain substances. Clinical chemistry analyzers can be used to evaluate a variety of samples, including, but not limited to, samples of serum, plasma, urine and/or other body fluids. Substances analyzed through these instruments include, for example, certain metabolites, electrolytes, proteins, and/or drugs. Various clinical chemistry analyzers are commercially available including the DxC® systems (Beckman Coulter, CA) and the AU® systems (Beckman Coulter, CA). In some aspects, the clinical chemistry analyzer is a high-throughput clinical chemistry analyzer.

In some aspects, the clinical chemistry analyzer may be used with an assay, such as an immunochemical assay. As used herein, the term “immunochemical assay” may be a laboratory method that utilizes chemical reactions between enzyme-conjugated antibodies or antigens to determine the amount of an analyte in a sample. In some aspects, the immunochemical assay is a colorimetric immunochemical assay, wherein a reaction between a chemical entity and an enzyme-conjugated antibody or antigen results in a detectable color change.

An “immunoassay analyzer” includes an automated analyzer that primarily relies on immune-based techniques (e.g. chemiluminescent or fluorescent immunoassays) to detect presence or concentration of an analyte in a solution. Immunoassay analyzers can be used to evaluate a variety of samples, including, but not limited to, samples of serum, plasma, urine and/or other body fluids. Substances analyzed through these instruments include, for example, protein biomarkers of reproductive health, cancer, cardiac health, and blood viruses. An “immunoassay analyzer” can include an instrument on which immunoassays have been automated. Various immunoassay analyzer are commercially available including the DxI® systems (Beckman Coulter, CA). In some aspects, the immunoassay analyzer is a high-throughput immunoassay analyzer.

As used herein, the term “immunoassay” may be a laboratory method that uses one or more antibodies or antigens to determine the amount of an analyte in a sample. It can be based on the interaction of antibodies with antigens, and because of the degree of selectivity for the analyte (either antigen or antibody), an immunoassay can be used to quantitatively determine very low concentrations of analyte in a test sample. In some aspects, the immunoassay is a luminescent immunoassay, wherein a reaction between a substrate and an enzyme-conjugated antibody or antigen results in a detectable luminescence. In some aspects, the luminescent immunoassay is a chemiluminescent assay.

Both the immunochemical assays and immunoassays may be “two-site” or “sandwich” assays which employ a first antibody or antibody fragment, which is described as the “capture” antibody, that is bound to a solid support, such as magnetic beads or particles disclosed herein, using procedures known in the art. Further, a second antibody or antibody fragment, which is described as the “detection” antibody, is coupled or conjugated with a label, such as the enzymes disclosed herein, using procedures known in the art. The label produces a detectable signal when it interacts with a substrate or substrates, so that the amount of signal measured corresponds to the amount of detection antibody that is bound to the analyte. Other types of immunochemical assays and immunoassays include competitive assays and antibody detection assays.

The terms “treat”, “treating”, or “treatment” refer to administering to a subject a compound or pharmaceutical composition to partially or completely alleviate, inhibit, ameliorate, or relieve the condition from which the subject is suffering. This means any manner in which one or more of the symptoms of a condition are ameliorated or otherwise beneficially altered. As used herein, amelioration of the symptoms of a particular condition refers to any lessening, whether permanent or temporary, lasting or transient, that can be attributed to or associated with treatment by the compounds, compositions, and methods of the present disclosure. For example, treating a subject can mean eliminating or reducing the clinical signs of a condition in the subject; arrest, inhibit, or slow the progression of the condition in the subject; and/or decrease the number, frequency, or severity of clinical symptoms of the condition in the subject. A “treatment protocol” is a protocol or regime developed regarding specific therapies (including pharmaceuticals or therapeutic interventions) for treatment. A “therapeutic intervention” refers to a clinical intervention intended to manage a disease, condition, disorder or injury and avoid further clinical interventions.

An “effective amount” includes a “therapeutically effective amount” and a “prophylactically effective amount.” The term “therapeutically effective amount” refers to an amount effective in treating and/or ameliorating a condition in a subject. The term “prophylactically effective amount” refers to an amount effective in preventing and/or substantially lessening the chances of a condition in a subject. The effective amount of the pharmaceutical composition may be administered orally or via intravenous injection. The exact amount required to achieve a therapeutically effective outcome will vary from subject to subject, depending on the species, age, and general condition of the subject, the severity of the condition, the particular composition, its mode of administration, its mode of activity, and the like.

The invention is defined in the claims. However, below is a non-exhaustive listing of non-limiting exemplary aspects. Any one or more of the features of these aspects may be combined with any one or more features of another example, embodiment, or aspect described herein.

### III. Assay Methods for Predicting the Likelihood of Developing a Neurodegenerative Disease

The use of ApoE to assess the pathology of neurodegenerative diseases, such as AD, has continued to evolve. The APOE (apolipoprotein) ε4 genotype is also linked to AD risk. The APOE gene has three common alleles ε2, 83, and ε4. APOE ε4 is associated with a significantly higher risk of developing AD. Having one copy is thought to increase a person's AD risk, while having two copies further elevates the risk. At the same time, the APOE ε4 genotype is also linked to a higher risk of developing of Amyloid-Related Imaging Abnormalities (ARIA) in patient's receiving amyloid-targeting disease modifying therapies (DMT) for AD. These abnormalities typically appear on MRI scans and come in two primary forms: ARIA-E (Edema), which is characterized by brain swelling (edema) or an increase in fluid in the brain's tissue, and ARIA-H (Hemosiderin), which involves small hemorrhages or bleeding in the brain, known as microhemorrhages. For example, Lecanemab, a monoclonal antibody targeting amyloid beta (Aβ) aggregates used for the treatment of AD in patients with mild cognitive impairment and confirmed amyloid pathology, has been found to be associated with ARIA-E and ARIA-H, with individuals with one or 2 copies APOE ε4 of having increased risk of such events. In some instances, candidates for a DMT having the APOE ε4 allele may require additional monitoring when receiving the DMT or may be precluded from receiving the DMT depending on other clinical factors.

Molecular testing, such as Polymerase Chain Reaction (PCR), is commonly used to determine the APOE genotype, including identifying the zygosity of the &4 allele, which is associated with AD risk and treatment-related complications. By amplifying specific regions of the APOE gene, PCR enables the detection of key polymorphisms that define the ε2, ε3, and ε4 alleles. Subsequent analysis, such as restriction fragment length polymorphism (RFLP) or sequencing, differentiates these alleles, allowing precise determination of whether an individual carries zero, one, or two copies of the ε4 allele. This information provides critical insights into genetic predispositions related to AD.

In the context of DMTs for AD, APOE genotyping via PCR is utilized to assess a patient's risk of developing ARIA. However, PCR-restriction fragment length polymorphism (RFLP) is labor intensive and prior to errors due to the number of steps. Assays, such as immunochemical assays and immunoassays, allow for the same genotyping as PCR, but have a much quicker run-time and are suitable for high-throughput analysis. Additionally, many of the diagnostic tools are limited to ascribing a risk of late-onset AD and cannot differentiate between medium and high AD population groups.

Methods that rely solely on ApoE4 levels to assess AD risk are limited by high patient variability, caused in part by confounding pathological and physiological conditions. These factors, such as coexisting neurodegenerative processes, vascular conditions, liver conditions, and individual differences in amyloid-beta metabolism, can obscure the relationship between APOE4 and AD risk, thereby reducing the accuracy and reliability of such diagnostic approaches. The comparison of an ApoE isomer, such as ApoE4, with the total ApoE present in a subject provides a greater predictor of the potential development and/or progression of a neurodegenerative disease than the use an ApoE isomer alone. It has been found that ApoE levels in serum are higher in subjects with liver cirrhosis as compared to healthy controls and subjects carrying the E3/E3 genotype are more likely to develop liver cirrhosis. It has also been found that subjects carrying the E4/E4 genotype have a higher risk of heart disease than healthy controls. By predicting, based on a ApoE4 comparison value (i.e., ratio ApoE4 to total ApoE), whether the subject is heterozygous or homozygous for the APOE ε4 allele, a clinician does not have to ascertain if there are confounding conditions which may skew the prediction if based solely on an ApoE genotype assessment.

The disclosed methods allow a user to rapidly determine a differentiation between ApoE4 heterozygous populations—which have roughly 3-fold increased risk of AD—from ApoE4 homozygous populations, which have roughly 12-fold increased risk of AD. In some aspects, the ApoE4 heterozygous populations carry an E3/E4 or an E2/E4 genotype (i.e., a single copy of each ApoE3 and ApoE4 or ApoE2 and ApoE4). In some aspects, the ApoE4 homozygous populations carry an E4/E4 genotype (i.e., two copies of ApoE4). An additional advantage of the disclosed a methods, is that, unlike PCR, it allows for direct confirmation of the expression of the E4 isoform.

In some instances, to assess a subject's Alzheimer's Disease status and zygosity for the APOE ε4 allele and assessing a degree of likelihood that the subject will be at risk of developing ARIA, the methods disclosed herein employ antibodies capable of binding to Apolipoprotein E (ApoE) and an apolipoprotein E4 isoform (ApoE4).

Affinity molecules include, but are not limited to, antibodies (including monoclonal antibodies, polyclonal antibodies, antibody fragments, synthetic antibody mimics, and the like), aptamers, affimers, DARPins, oligonucleotides, peptides, and antigens.

As used herein, the term “antibody” or “antibodies” refers to a binding protein, immunoglobulin, or glycoprotein that maintains antigen-binding properties. An antibody often comprises a variable domain and a constant domain in each of a heavy chain and a light chain. Accordingly, most antibodies have a heavy chain variable domain (VH) and a light chain variable domain (VL) that together form the portion of the antibody that binds to the antigen. Within each variable domain are three complementarity determining regions (CDR) which form loops in the heavy chain variable domain (VH) and light chain variable domain (VL) that contact the surface of the antigen. Antibodies herein also include “antigen binding portion” or fragments of the antibody that are capable of binding to the antigen.

As used herein, the term “epitope” refers to a binding site recognized by an antibody. Epitopes may include any molecule or grouping thereof, including, but not limited to, amino acid side chains, sugars, and lipids, and can have a specific three-dimensional structure or conformation.

One aspect of the disclosure is an method of assessing a subject's likelihood of developing a neurodegenerative disease. In an embodiment, the method includes contacting a first portion of at least one biological sample from the subject with first and second apolipoprotein (ApoE) antibodies, wherein the second ApoE antibody is specific to a single apolipoprotein E isoform, generating a first reaction mixture. Non-limiting examples of the biological sample include serum, whole blood, plasma, and/or cerebral spinal fluid. A detection reaction is conducted in the first reaction mixture generating a first detection signal, which is then detected. A second portion of the at least one biological sample from the subject with third and fourth ApoE antibodies capable of binding to multiple ApoE isoforms, wherein the third or fourth ApoE antibody and the first ApoE antibody bind to a same epitope of ApoE, generating a second reaction mixture. A detection reaction is conducted in the second reaction mixture generating a second detection signal, which is detected. A ratio of the first detection signal to the second detection signal is calculated and the subject is assigned a degree of likelihood that the subject will develop a neurodegenerative disease and/or an accuracy of diagnosis of a neurodegenerative disease based on comparison of the ratio to a ratio established in a reference population.

One aspect of the disclosure is a method of a method of assessing a degree of likelihood that a subject will be at risk of developing ARIA. In an embodiment, the method includes contacting a first portion of at least one biological sample from the subject with first and second apolipoprotein (ApoE) antibodies, wherein the second ApoE antibody is specific to apolipoprotein E4 isoform (ApoE4), generating a first reaction mixture. A detection reaction is conducted in the first reaction mixture generating a first signal, which is then detected. A second portion of the at least one biological sample from the subject is then contacted with third and fourth ApoE antibodies capable of binding to multiple ApoE isoforms, wherein the third or fourth ApoE antibody and the first ApoE antibody bind to a same epitope of ApoE, generating a second reaction mixture. A detection reaction is conducted in the second reaction mixture generating a second signal, which is then detected. A ratio of the first signal to the second signal is calculated and the subject is assigned an APOE ε4 genotype based on comparison of the ratio to a ratio established in a reference population and then assessed a degree of likelihood that the subject will be at risk of developing ARIA based on the whether the subject is heterozygous or homozygous for the APOE ε4 allele.

A novel aspect of the methods disclosed are they employ a panel immunoassay, which is a laboratory technique used to simultaneously measure multiple analytes within a single sample. As disclosed herein, the methods allow for inclusion of several analytes to be assessed in parallel, enabling a simultaneous analysis of ApoE isoforms and total ApoE. In some aspects, this allows for a comprehensive analysis of Alzheimer's disease status and zygosity for the APOE ε4 allele in one assay.

The disclosed methods provide distinct advantages to several methodologies used to assessing a subject's likelihood of developing a neurodegenerative disease, including diagnosing AD, such as PCR, cerebrospinal fluid (CSF) analysis, and positron emission tomography (PET) scans. Each of these approaches presents distinct disadvantages that can impact their effectiveness and applicability in clinical settings.

PCR, while effective for genotyping, does not provide information on the actual expression levels of the APOE4 protein, potentially missing functional insights important for understanding disease risk. Additionally, there is a lack of standardization across different assays, which can affect the reliability and comparability of results between laboratories.

The procedure to obtain CSF is invasive, requiring a lumbar puncture that can cause discomfort, pain, and carries risks such as headaches or infections. This invasiveness may limit the willingness of patients to undergo frequent testing, which is often necessary for monitoring disease progression or treatment efficacy. Moreover, CSF collection requires specialized medical settings and trained personnel, making it less accessible and more costly compared to blood-based assays. Variability in CSF biomarker levels due to factors like hydration status and diurnal fluctuations can also impact the consistency and reliability of results.

PET scans are expensive and require access to specialized imaging facilities, limiting their availability to larger medical centers and increasing the overall cost of diagnosis. Additionally, PET scans involve exposure to radioactive tracers, raising concerns about radiation safety, especially with repeated scans needed for longitudinal studies or treatment monitoring. The procedure is time-consuming and may be uncomfortable for patients, particularly those with cognitive impairments or movement difficulties. Furthermore, interpreting PET scan results requires highly trained specialists, which can introduce variability and subjectivity in diagnosis.

These disadvantages highlight the need for a multimodal diagnostic approach to enhance accuracy and reliability in assessing a subject's likelihood of developing a neurodegenerative disease and as well as assessing zygosity for the APOE ε4 allele and a degree of likelihood that the subject will be at risk of developing ARIA.

In an embodiment, a first portion of at least one biological sample from a subject is exposed to a first capture antibody capable of binding ApoE or capable of recognizing an epitope of ApoE, and a first detector affinity molecule specific to a single isoform of apolipoprotein E4, generating a first reaction mixture. The first detector affinity molecule specific to the single isoform of ApoE may be specific to an epitope of an ApoE isoform. A detection reaction is conducted in the first reaction mixture wherein the first detector affinity molecule generates a first detection signal, which is then recorded. In some aspects, the detection reaction is a chemiluminescent reaction, an electrochemiluminescence reaction, an electrogenerated chemiluminescence reaction, a photoluminescence reaction, or a bioluminescence reaction. In some embodiments, the chemiluminescent reaction is a dioxetane-based reaction, a luminol-based reaction, a acridinium ester-based reaction, a peroxyoxalate reaction, a luciferin-luciferase reaction, a metal-catalyzed reaction, a halogen-based reaction, or a hydrazine-based reaction. In some embodiments, the detector affinity molecule is conjugated to an enzyme, such alkaline phosphatase or horseradish peroxidase. In this embodiment, a substrate for the enzyme is added to the second reaction mixture triggering a chemiluminescent reaction.

In an embodiment, a second portion of the at least one biological sample from the subject is exposed to a second capture antibody capable of binding ApoE or capable of recognizing an epitope of ApoE and a second detector affinity molecule capable of binding to multiple isoforms of ApoE, generating a second reaction mixture. In some embodiments, the first capture antibody and the second capture antibody bind to or recognize the same epitope of ApoE. In some embodiments, the first capture antibody and the second capture antibody are the same. A detection reaction is conducted in the second reaction mixture wherein the second detector affinity molecule generates a second detection signal, which is then recorded. In some aspects, the detection reaction is a chemiluminescent reaction, an electrochemiluminescence reaction, an electrogenerated chemiluminescence reaction, a photoluminescence reaction, or a bioluminescence reaction. In some embodiments, the chemiluminescent reaction is a dioxetane-based reaction, a luminol-based reaction, a acridinium ester-based reaction, a peroxyoxalate reaction, a luciferin-luciferase reaction, a metal-catalyzed reaction, a halogen-based reaction, or a hydrazine-based reaction. In some embodiments, the detector affinity molecule is conjugated to an enzyme, such alkaline phosphatase or horseradish peroxidase. In this embodiment, a substrate for the enzyme is added to the second reaction mixture triggering a chemiluminescent reaction.

A ratio of the recorded first detection signal to the recorded second detection signal is calculated to generate an ApoE isomer comparison value. In an aspect, if the ApoE isomer is ApoE2, the comparison value generated is an ApoE2 comparison value. In an aspect, if the ApoE isomer is ApoE3, the comparison value generated is an ApoE3 comparison value. In an aspect, if the ApoE isomer is ApoE4, the comparison value generated is an ApoE4 comparison value.

The ApoE4 comparison value (APOE4 relative to total APOE) is indicative of genotype as the APOE4 is expressed by &4 homozygous and heterozygous individuals. The ApoE4 comparison value is compared to a ratio established in a reference population and can be used to distinguish between &4 homozygous and heterozygous individual by, for example, determining if a significant amount of another isoform is being expressed or by controlling for another condition, such as coexisting neurodegenerative processes, vascular conditions, liver conditions, and individual differences in amyloid-beta metabolism.

In some aspects, if an ApoE4 comparison value is between about 0 and about 0.5, the subject likely has an ApoE2/ApoE2, ApoE2/ApoE3, or ApoE3/ApoE3 genotype. In these aspects, the risk of developing AD is low.

In some aspects, if an ApoE4 comparison value is between about 0.5 and about 1.0, the subject likely has an ApoE2/ApoE4 or ApoE3/ApoE4 genotype. In these aspects, the risk of developing AD is medium.

In some aspects, if an ApoE4 comparison value ratio is between about 1.0 or greater, the subject likely has an ApoE4/ApoE4 genotype. In these aspects, the risk of developing AD is high.

Based on the comparison, a prediction may also be made whether the subject is heterozygous or homozygous for the APOE ε4 allele. In some embodiments, the method further comprises assessing a degree of likelihood that the subject will be at risk of developing ARIA based on the whether the subject is heterozygous or homozygous for the APOE ε4 allele. In some embodiments, the degree of likelihood that the subject will develop ARIA is low risk, medium risk, or high risk.

The presence of ApoE4 is also indictive of an increased risk of developing AD in a gene dose-dependent manner. Without being limited by any theory, it is thought that individuals with one copy of the ApoE4 gene have about three times greater the risk of developing AD as compared to individuals without ApoE4 present in their blood. While individuals with two copies of the ApoE4 gene have about a twelve-fold increased risk of developing AD, as compared to individuals without ApoE4 present in their blood.

In some embodiments, if the first detection signal is equal to or less than a reference signal established in a low risk reference population and the subject is assigned a low risk degree of likelihood, the second step of the assay method may not be completed. However, in some aspects, the second step of the assay method is completed and the calculated ratio is used to correlate the low risk degree assignment.

In some embodiments, if the first detection signal is higher than a reference signal established in a low risk reference population and the subject is assigned a medium or high risk degree of likelihood, the second step of the assay method is completed and the calculated ratio may be used to correlate the medium or high risk degree of likelihood assignment.

In some aspects, assigning the subject a degree of likelihood that the subject will develop a neurodegenerative disease includes assessing the subject's Alzheimer's disease status. In some aspects, assessing the subject's Alzheimer's disease status comprises assessing a degree of likelihood that the subject will develop Alzheimer's disease. For example, in non-limiting aspects, the degree of likelihood that the subject will develop Alzheimer's disease may be classified as low risk, medium risk, or high risk. In some aspects, the method may further include assessing the subject's Alzheimer's Disease status by analyzing a subject's levels of amyloid protein detected through a PET scan of the brain and/or to the levels of tau and/or beta-amyloid proteins in CSF and correlating the analyzed levels to a reference population. In some embodiments, the correlations includes additional clinical factors such as a degree of cognitive decline or a level of cognitive impairment.

The complex and specific information provided by the disclosed assay methods may become a critical feature in individualized therapeutics. As such, a clinician may use this information to better inform their treatment, intervention, and/or monitoring efforts of a subject.

In some aspects, assessing a subject's likelihood of developing a neurodegenerative disease may also include assessing additional clinical factors such as a degree of cognitive decline or a level of cognitive impairment or the presence of the APOE4 genotype. In some embodiments, the assessment is used to identify patients having AD, or at risk of progressing to AD; detect abnormal amyloid pathology; monitor the treatment responses to clinical and/or pharmaceutical interventions; and/or monitor the progression of AD. In some embodiments, the assessment is used to identify patients who have a low risk, medium risk, or high risk of developing or progressing to AD.

In some aspects, a clinician may use the disclosed method to determine a treatment selection and prognosis. If the subject is diagnosed with Alzheimer's disease, the subject may recommended for further treatment. The further treatment may include qualifying the subject for a clinical trial, assessing the risks associated with the subject entering the clinical trial, and/or increasing safeguards for the subject in clinical trial.

In some aspects, further treatment may include assessing a subject's eligibility for a disease-modifying therapy (DMT), administering a DMT and/or providing monitoring or additional treatments in conjunction with the DMT. Rather than just treating the symptoms, DMTs for Alzheimer's disease aim to slow down the progression of the disease. Non-limiting examples of DMT include anti-amyloid therapies, which include monoclonal antibody therapies such as Aducanumab, Lecanemab, and Donanemab; anti-tau-targeted therapies, which are being developed to prevent the spread of tau tangles and disrupt neuronal function in Alzheimer's; immunotherapies such as vaccines; beta-site amyloid precursor protein cleaving enzyme (BACE) inhibitors, which aim to reduce the production of amyloid-beta; anti-inflammatory drugs, including those that target glial cells (which become overactive in Alzheimer's) or drugs that reduce general neuroinflammation; and neurotrophic drugs, which include Brain-Derived Neurotrophic Factor (BDNF) mimetics, Nerve Growth Factor (NGF) analogs, Glial-Derived Neurotrophic Factor (GDNF) agents, and neuroprotective drugs.

In some aspects, the method includes assessing a degree of likelihood that the subject will be at risk of developing ARIA after the administration of or treatment with certain DMTs (such as anti-amyloid therapies) and includes analyzing a subject's ApoE4 comparison value and determining whether the subject is heterozygous or homozygous for the APOE ε4 allele. In some aspects, the comparison value is correlated to a reference population.

In an embodiment, if degree of likelihood that the subject will develop ARIA is medium risk or high risk and the subject is diagnosed with Alzheimer's disease, and the subject is recommended for further treatment, wherein the further treatment may include administration of a DMT with increased monitoring. In some embodiments, the further treatment may include administration of an anti-amyloid therapy with increased monitoring as long as the subject is also not taking anti-coagulants.

In some aspects, a clinician may use the disclosed methods to inform decisions regarding the recommendation or administration of clinical or therapeutic interventions. In a non-limiting aspect, if a subject is assigned a medium or high risk of developing or progressing to AD, a clinician may recommend multimodal interventions including, but not limited to, improved nutrition, increased physical activity, cognitive engagement, and management of comorbidities or improved cognitive functioning.

In some aspects, a clinician may use the disclosed assays to monitor the clinical progression of AD. In this embodiment, the disclosed assay may be used on a biological sample from a subject taken at different time points. In an aspect, at least a first sample and a second sample may be collected at least several days, alternatively at least several months, alternatively at least several years apart. However, it is appreciated that any time point or number of samples may be analyzed.

In some aspects, a clinician may use the disclosed assays to monitor the clinical progression of a neurological disease or disorder. In this embodiment, the disclosed assay may be used on a biological sample from a subject taken at different time points. In an aspect, at least a first sample and a second sample may be collected at least several days, alternatively at least several months, alternatively at least several years apart. However, it is appreciated that any time point or number of samples may be analyzed.

A novel aspect of the disclosure is that two APOE sandwich immunoassays performed as the steps of the APOE4 genotyping assay can be combined into a single workflow and can use the same capture reagent. In some cases, calibration of the APOE immunoassays is not necessary because they are performed as part of the same workflow and leverage the same capture antibody. The signals produced by each immunoassay can be compared to generate a ratio indicative of APOE genotype and without being converted to concentration values using a calibration curve. In one embodiment the capture antibodies and detection antibodies are combined into a single reagent pack. A “reagent pack” may include any suitable container that can store a reagent. An example of a reagent pack can include a generally rectangular elongated body formed to include multiple reagent vessels including one or more large reagent vessels, and one or more relatively smaller reagent vessels, as well as features to facilitate handling and automation. U.S. Pat. No. 9,519,000, which is incorporated by reference in its entirety herein, discloses non-limiting examples of a reagent pack that may be used in an aspect of the invention.

In some instances, using separate APOE and APOE4 assays that are not combined into a single workflow with a shared antibody may introduce variance into the analysis relative to the present assay (e.g., lot-to-lot variability) which may affect the specificity, sensitivity, and accuracy of the measurement. It also increases the run time, lowering the throughput of the assay. In addition to overcoming the problems with separate assays, this combined assay is robust, highly accurate, inexpensive, does not require a calibrator, and requires less run time than conventional ApoE4 or p-tau assays, allowing a user to rapidly analyze a biological sample.

For example, in conventional assays methods, a calibration curve is employed to quantify the analyte present in the sample. In some instances, to prepare a calibration curve, a one or more preparations (e.g., calibrator levels) containing known concentrations of the antigen to be measured are required. The signal produced by each preparation is plotted against the known concentration of antigen in each preparation, and a curve is generated from the data points using a math model. When the assay is performed on a sample having an ApoE4 analyte, the calibration curve can be used to correlate the signal produced by the analyte to a concentration. The calibration curve must be constructed before running the assay. In some instances, a calibration curve for an particular assay must be reconstructed after a certain time period has elapsed or amount of assays have been performed. The disclosed combined assay methods do not require a calibrator because they are not measuring the concentration of an analyte, but ratio of signals produced by each analyte. An advantage of the disclosed assay methods includes the elimination of calibrant interference, while providing a faster, more precise, and reproducible method for determining an ApoE isoform: total ApoE ratio. In exemplary embodiments, an ApoE4: total ApoE ratio can be used to distinguish between ApoE4 heterozygous populations and ApoE4 homozygous populations. In these embodiments, the total ApoE antibody serves as an internal control, reducing the potential ApoE4 detection variability.

A “reagent vessel” may refer to a vessel, unit, fluid container, well, or the like that is configured to store reagents. In some aspects, the reagent pack comprises enough reagent vessels to perform a combined assay for total ApoE and ApoE4 and/or ApoE3 and/or ApoE2. In some embodiments, the reagent pack further includes containment walls arranged between the reagent vessels. In an aspect, the reagent pack may include at least one reagent vessel, alternatively at least two reagent vessels, alternatively at least three reagent vessels, alternatively at least four reagent vessels, alternatively at least five reagent vessels, or alternatively at least ten reagent vessels. In an embodiments, each reagent vessel is configured to store a volume of reagent required for at least one instance of the assays, wherein at least one reagent comprises the first capture antibody, at least one reagent comprises the first detector affinity molecule, and at least one reagent comprises the second detector affinity molecule.

Each reagent vessel may be large enough to accommodate a microtip or disposable tip (i.e., dispo-tip) of a reagent pipettor used to remove a volume of reagent for use in an assay. In certain embodiments, the reagent pack may be maintained at a temperature of between about 4 °C to 10 °C.

In an embodiment, the reagent pack may be configured to accommodate sufficient volumes of reagents for multiple instances of an assay. In some embodiments, each reagent pack includes reagents for about 20 to about 100 instances of an assay and in some cases about 50 instances.

In an embodiment, the reagent vessels include an elastomeric self-sealing membrane. An elastomeric self-sealing membrane may be a polymer, such as polypropylene, which is able to regain its original shape when pierced. For some embodiments, the elastomeric membrane can be a thermoplastic elastomer with hardness of 30-40 durometer (Shore) A. In other embodiments, the hardness can be 20-50 (Shore) A, or about 30 (Shore) A. Elastomers deform sufficiently to form a tight seal with the vessel base. Thermoplastic elastomers are advantageous because of their compatibility with plastics injection molding processes.

The elastomeric membrane can be large enough to provide adequate compression without bottoming on the sealing portion of the vessel. The hardness and dimensions can cooperate to allow the elastomeric membrane to the sealing portion with reasonable sealing force. In some embodiments, the elastomeric membrane diameter is small enough so that, when compressed by engagement of the pipettor tip, it conforms to the sealing portion without contacting the wall of the pipettor tip. This advantageously concentrates sealing force to the sealing portion of vessel and distributes sealing force evenly to prevent leaks. In some embodiments, the sealing force is about 44 newtons (about 9.9 lbs.) and produces a pressure on the sealing surface of about 300 (about 43.5 pounds per square inch) to about 1000 kPa (145.0 pounds per square inch).

In an aspect, the capture antibody is conjugated to a magnetic bead or a magnetic particle. In some instances, magnetic beads (also known as magnetic particles, paramagnetic particles, or superparamagnetic particles) consist of a polystyrene core surrounded by a thin layer of small iron oxide particles (~20-30 nm), such as magnetite. On the surface, the magnetic beads are encapsulated by, for example, a polymer, protein A, protein G, protein L, a secondary antibody, or an epoxy. Surface modification of the coating minimizes any non-specific protein binding. Antibodies targeting the analyte of interest or capture antibody can be covalently coupled to the surface of the magnetic bead. In some embodiments, there is about 2 μg or antibody per mg of magnetic bead. In some embodiments, there is about 3 μg or antibody per mg of magnetic bead. In some embodiments, there is about 4 μg or antibody per mg of magnetic bead. In some embodiments, there is about 5 μg or antibody per mg of magnetic bead. In some embodiments, there is about 6 μg or antibody per mg of magnetic bead. In some embodiments, there is about 7 μg or antibody per mg of magnetic bead. In some embodiments, there is about 8 μg or antibody per mg of magnetic bead. In some embodiments, there is about 9 μg or antibody per mg of magnetic bead. In some embodiments, there is about 10 μg or antibody per mg of magnetic bead. In some embodiments, there is about 12 μg or antibody per mg of magnetic bead. In some embodiments, there is about 13 μg or antibody per mg of magnetic bead. In some embodiments, there is about 14 μg or antibody per mg of magnetic bead. In some embodiments, there is about 15 μg or antibody per mg of magnetic bead.

In some aspects, the first detector affinity molecule and/or the second detector affinity molecule is conjugated to an enzyme, such as alkaline phosphatase or horseradish peroxidase. In certain embodiments, the affinity molecule is an enzyme-conjugated antibody, an enzyme-conjugated antigen, an alkaline phosphatase (ALP)-conjugated antibody, or a labeled antibody. In some aspects, the first detector affinity molecule is anti-ApoE4-ALP. In some aspects, the first detector affinity molecule is anti-ApoE2-ALP. In some aspects, the first detector affinity molecule is anti-ApoE3-ALP. In some aspects, the second detector affinity molecule is anti-ApoE-ALP. In some aspects, the detector affinity molecule is an antibody, a monoclonal antibody, a polyclonal antibody, an antibody fragment, a synthetic antibody mimic, an aptamer, an affimer, DARPins, oligonucleotide, peptide, or antigen.

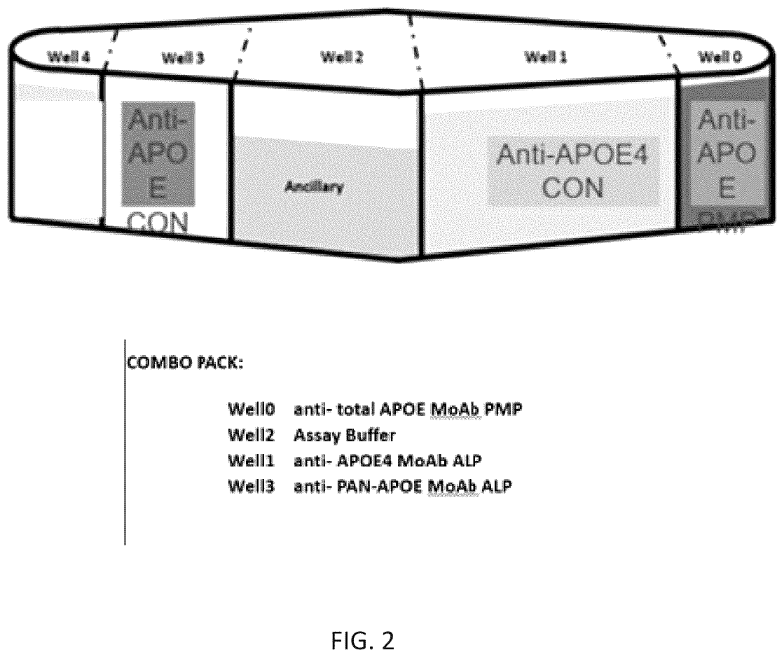

In an exemplary embodiment, as depicted in FIG. 2, the reagent pack may comprise at least 4 reagent vessels, wherein each reagent vessel includes sufficient volumes of reagents for multiple instances of an assay. In an aspect, a first reagent vessel may include a first reagent formulation which comprises a ApoE capture antibody (anti-ApoE). The ApoE antibody may be conjugated to a magnetic bead or magnetic particle. A second reagent vessel may include a second reagent formulation which comprises a first detector affinity molecule. In an embodiment, the first detector affinity molecule is an ApoE4 antibody (anti-ApoE4) conjugated to ALP. In an embodiment, the first detector affinity molecule is an ApoE2 antibody (anti-ApoE2) conjugated to ALP. In an embodiment, the first detector affinity molecule is an ApoE3 antibody (anti-ApoE3) conjugated to ALP. A third reagent vessel may include buffers and/or salts needed for the methods disclosed herein. A fourth reagent vessel may include a third reagent formulation which comprises a second detector affinity molecule. In an embodiment, the second detector affinity molecule is an ApoE antibody conjugated to ALP. In an aspect, the first detector molecule and second detector molecule bind a different binding sites than the capture antibody.

“Anti-PAN-ApoE”, “PAN-ApoE antibody”, “anti-total-ApoE”, and “total-ApoE antibody” are used interchangeably herein, and refer to an ApoE antibody which recognizes endogenous levels of total ApoE protein. In certain embodiments, the ApoE antibody also recognizes overexpressed ApoE2, ApoE3 and ApoE4 proteins.

In an aspect, the first detection signal and/or the second detection signal are generated via a detection reaction between the first detector affinity molecule and/or the second detector affinity molecule and a substrate formulation. Depending on the analysis desired, a first substrate formulation may be added to the first reaction mixture, and a second substrate formulation may be added to the second reaction mixture. In some aspects, the first substrate formulation and the second substrate formulation are the same.

In an aspect, the detection signal may be detected using a detector, such as a luminometer, an electrochemiluminescence (ECL) detector, a photomultiplier Tube (PMT) detector, photometer, a fluorometer, or a bioluminescence detector.

In an aspect, the substrate formulation is configured to produce a colorimetric response. These substrates produce a visible color based on a chemical reaction between an analyte and a reagent. At operation, this substrate is added to a vessel with a reaction mixture and the light generated is measured with a photometer.

In an aspect, the substrate formulation is configured to produce chemiluminescence. These substrates can produce light and thereby provide detection corresponding to a quantity of analytes captured. The term “chemiluminescent compound” refers to a compound that produces chemiluminescence in the presence of a phosphatase enzyme and oxygen under appropriate conditions as provided herein. Chemiluminescent compounds useful in the present formulations are capable of generating chemiluminescence when contacted with an alkaline phosphatase.

In a non-limiting example, the substrate formulation includes a chemiluminescent compound of formula I or a salt thereof:

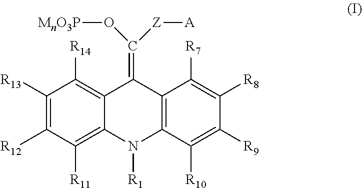

wherein
A is C₁₋₆ haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁₋₆ alkyl, C₁₋₆ haloalkyl, and C₅₋₁₄ aralkyl groups;
R₇-R₁₄ are independently H, C₁₋₆ alkoxy, halo, C₁₋₄ alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2;
a cationic aromatic compound (CAC);
a background reducing agent; and
an ether-linked nonionic surfactant or a hydrophilic polymer.

Chemiluminescent compounds useful in the present formulations are capable of generating chemiluminescence when contacted with an alkaline phosphatase. Such compounds can be synthesized as described in U.S. Pat. Nos. 6,45,727, 6,90,571, 6,139,782, 6,218,137, 6,270,695, 6,296,787, and 10,703,971 each of which is incorporated by reference herein.

At operation, this substrate is added to a vessel with a reaction mixture and light generated by the reaction is measured with a luminometer. In some aspects, the first substrate formulation and the second substrate formulation are the same.

In an embodiment, the detector can generate an output signal that can be processed to generate a relative light unit (“RLU”) value (i.e., an output response) indicating a result of the assay. For example, a larger RLU value indicates more light, which indicates a larger amount of the analyte in the biological sample than a smaller RLU value indicates.

In an exemplary method of producing light from the reaction of the chemiluminescent substrate with a phosphatase enzyme (e.g., detection antibody), the reaction is performed at a temperature between 5 °C and 50 °C, preferably between 20 °C and 40 °C in an aqueous buffer solution at a pH between 7 and 12, 8 and 11, or preferably between 8.5 and 10. The enzyme is preferably an alkaline phosphatase or an alkaline phosphatase conjugate.

In one aspect, the substrate formulation comprises 0.01 mM-50 mM compound I, 0.01-200 μM cationic aromatic compound, 1 μM-10 mM background reducing agent, 0.05-20 g/L ether-linked non-ionic surfactant or hydrophilic polymer, 0.01-10 g/L anionic surfactant, and an amine buffer at from 0.025M to 0.65M and at pH 7-12.

In one aspect, the substrate formulation comprises 0.05 mM-10 mM compound I, 0.05-50 μM cationic aromatic compound, 10 μM-1000 μM background reducing agent, 0.1 to 10 g/L ether-linked non-ionic surfactant or hydrophilic polymer, 0.1 to 5 g/L anionic surfactant, and an amine buffer at from 0.05M to 0.5M and at pH 8-11.

In one aspect, the substrate formulation comprises 0.1 mM-5 mM compound I, 0.1-25 μM cationic aromatic compound, 50 to 500 μM background reducing agent, 0.2 to 5 g/L ether-linked non-ionic surfactant or hydrophilic polymer, 0.1 to 5 g/L anionic surfactant, and an amine buffer at from 0.1M-0.4M and at pH 8-11.

In one aspect, the compound I has the formula

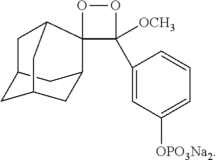

### IV. Application of Calibration Independent Immunoassays to Other Systems

The methods described herein for assessing the relative presence of a subgroup or subpopulation of an analyte can be applied to systems outside of ApoE as well. The methods are applicable to systems where an analyte subpopulation is distinguishable from the total analyte population (or another analyte subpopulation) and the ratio of the pair provides qualitative or quantitative information about the sample. Conditions related to cardiac, hepatic, urinary, bile, adrenal, neurological, immunological, reproductive, inflammatory, metabolic, digestive, musculoskeletal, respiratory, endocrine, lymphatic, integumentary systems can potentially be assessed via the methods described herein. Specific conditions include allergies, inflammatory responses, prostate or breast cancer, sepsis, infectious diseases, heart failure, diabetes, and bone metabolism.

In a first embodiment, the first analyte subgroup differs from the other analyte subgroups by a post-translational modification or a proteolytic cleavage event. In such embodiments, it may be advantageous if the analyte is a peptide, polypeptide, protein, enzyme, or antibody. The post-translational modification can be, for example, phosphorylation, glycosylation, ubiquitination, S-nitrosylation, methylation, N-acetylation, or lipidation. Proteolytic cleavage may be, for example, by serine, cysteine, or aspartic acid proteases proteases, or by zinc metalloproteases. The methods described herein can compare, for example, truncated to untruncated forms of an analyte, phorphorylated to unphosphorylated forms, truncated forms that differ in truncation, or combinations of one or more of these modifications.

In some embodiments, the first analyte subgroup comprises a full-length phosphorylated form of the protein and the at least one additional analyte subgroup comprises a full-length non-phosphorylated form of the protein. In some embodiments, the first analyte subgroup comprises a tau protein phosphorylated at a residue selected from Thr181, Ser198, Ser199, Ser202, Thr217, Thr231, Ser262, Ser396, or Ser422. In such embodiments, the total analyte comprises all tau proteins or the at least one additional analyte subgroup comprises a tau protein unphosphorylated at a residue, wherein the unphosphorylated residue is the same residue as in the first analyte subgroup.

In another embodiment, the first analyte subgroup and the at least one additional analyte subgroup correspond to protein isoforms encoded by different alleles of a gene. In such embodiments, the comparison of the first and second signals is indicative of a subject's genotype. For example, in an embodiment, the analyte comprises a haptoglobin isoform selected from genotypes Hp1-1, Hp2-1, and Hp2-2.

In another embodiment, the first analyte subgroup and the at least one additional analyte subgroup comprises a normal protein and a mutant or aberrant form of the protein resulting from a single of multiple-gene disorder. Alternatively, the first analyte subgroup and the at least one additional analyte subgroup comprises protein isoforms encoded by allelic variants of a gene that encodes a drug-metabolizing enzyme wherein differing enzymatic activity results from underlying genetic polymorphism.

In some embodiments, the first analyte subgroup differs from the other analyte subgroups due to conformational changes in the analyte structure. For example, the first analyte subgroup may comprise mis-folded proteins or mis-aligned polypeptide structures in the analyte.

Use of the methods and systems described herein across a variety of analytes is useful for diagnostic purposes. The methods can be utilize in assigning a genotype, assessing a biomedical diagnostic value, diagnosing a condition, staging a disease, determining a prognosis, detecting a medical deviation, or determining a treatment in the subject based on the relative presence of the first analyte subgroup compared to the total presence of analyte or the at least one additional analyte subgroup.

### V. Kits Incorporating the Elements of Assay

The reagents needed to do the methods described herein can be incorporated into a kit that provides for ease of use and reliability. The kit may incorporate a first reagent set to react with the sample and provide a first quantitative signal. In some embodiments, the first reagent set comprises a first capture antibody that binds to all forms of the analyte and a first detector affinity molecule that is specific to a particular first analyte. The kit may also incorporate a second reagent set to react with the sample and provide a second quantitative signal. In some embodiments, the second reagent set comprises a second capture antibody that binds to all forms of the analyte and a second detector affinity molecule that binds to all forms of the analyte or is specific to a particular, second analyte that is different from the first analyte.

The first and second capture antibodies may be the same or different and may bind to the same epitope on the analyte. In some embodiments, first and second capture antibodies may be bound to a bead or particle, wherein the particle may be a magnetic particle. The detector affinity molecules can be an antibody, a monoclonal antibody, a polyclonal antibody, an antibody fragment, a synthetic antibody mimic, an aptamer, an affimer, DARPins, oligonucleotide, peptide, or antigen. In some embodiments, the detector affinity molecules are conjugated to an enzyme for detection purposes, such as for use in chemiluminescence as described herein.

The kit may further comprise instructions on how to use the kit, wherein the instructions may be in an electronic form that is readable via the internet or a display on an instrument. The kit may be incorporated into one or more reagent packs that can be used on an automated immunoassay instrument. In such embodiments, the instructions for use may be an electronic file that is readable by the instrument.

Kits may further comprise components including buffers, buffer containers, fluid collection means, a sample collection apparatus, control samples, control particles, capture reagents, tools to assist in collecting of samples to be assessed for the analyte of interest (e.g., swabs), disposal apparatus (e.g., biohazard waste bags), and/or other information or instructions regarding the sample collection apparatus (e.g., lot information, expiration date, etc.).

### VI. Automated Analyzer

In an aspect, an automated analyzer disclosed herein includes the following basic structural and functional modules: a sample presentation unit, an analytic unit, an incubator station, a washing station, a read station, and reagent storage. In addition, the automated analyzer may include a pipettor arrangement with at least one sample pipettor and at least one reagent pipettor, and at least one transport device. In an aspect, the transport device includes mechanisms, such as pick-and-place grippers, which are used to transport sample and reaction vessels among the various modules of the automated analyzer.

The automated analyzer may include a container carriage device that is configured to hold and carry the containers at various locations in the instrument so that the analytic unit, incubator station, wash station, and read station can use the containers in various manners. Examples of container carriage devices include vessel racks (e.g., a sample rack, a reagent rack, and a diluent rack), the sample presentation unit, vessel carriage units (e.g., a sample carriage unit, a reaction vessel carriage unit, and a reagent carriage unit), vessel transfer units (e.g., a sample transfer unit, a reagent transfer unit, an incubator transfer unit, and an reaction vessel transfer unit), and vessel holding plates or wheels (e.g., a sample wheel, an incubator, and a wash wheel), which are described herein.

The read station may include a detector arrangement. In an aspect, the detector arrangement may include a detector that is configured to detect colorimetric response, light or luminescence. The detector may be a photometer, luminescence detector, a chemiluminescence detector, a luminometer, or a photomultiplier-based detection instrument.

In an embodiment, the detector includes a photometer (or photometry unit) configured to emit light of a predetermined wavelength toward a reaction vessel and receive the light that transmits through the reaction mixture. In an embodiment, the detector includes a light detector configured to sense photons emitted from assay reactions over a period of time, an analog circuit configured to provide an analog signal based on the photons emitted from the assay reactions over the period of time, and a counter circuit configured to provide a photon count based on the photons emitted from the assay reactions over the period of time. U.S. Pat. Nos. 5,14,216 and 11,604,146, which are incorporated by reference in its entirety herein, discloses non-limiting examples of a detector that may be used in an aspect of the invention.

In an aspect, the analytic unit is configured to receive and analyze samples. In an aspect, the analytic unit configured to perform an assay, such as an immunochemical assay or immunoassay. In certain embodiments, the analytic unit includes the pipettor arrangement. The pipettor arrangement may be configured to aliquot, aspirate, and dispense fluidic substances into various vessels, including, but not limited to sample vessels, diluent vessels, reagent vessels, and reaction vessels. Fluidic substances are substances that have fluidic characteristics. In some embodiments, the fluidic substance is a single fluidic substance. In other embodiments, the fluidic substance is a mixture of a plurality of substances.

The pipettor arrangement may contain at least one, two, three, or four reagent pipettors used to mix reagents with sample aliquots for an assay. The pipettor arrangement may also contain at least one, two, three, or four sample pipettors used to transfer sample aliquots for an assay. In some instances, the pipettor arrangement contains one sample pipettor. In some instances, the pipettor arrangement contains one sample pipettor and four reagent pipettors. In an aspect, the reagent pipettors may be arranged as dual reagent pipetting stations and are independent to each other, each having its own fluid pumps and valves, wash towers, reaction vessel carriages, and pipettor. A sample aliquot may be transferred from a sample retention vessel into a reaction vessel using the sample pipettor in order to mix the sample aliquot with one or more reagents. In an aspect, the at least one reagent pipettor and at least one sample pipettor are configured to aspirate and/or dispense less than about 10 μL.

In an aspect, the first reagent pipettor, the second reagent pipettor, the third reagent pipettor, and/or the fourth reagent pipettor are selectively and/or simultaneously operated. In certain embodiments, the sample pipettor, the first reagent pipettor, the second reagent pipettor, the third reagent pipettor, and/or the fourth reagent pipettor are configured to engage a dispense tip prior to aspiration.

The disclosed simultaneous and/or selective operation of the pipettors allows for a high-throughput analysis. In some embodiments, the method is configured to analyze at least about 200 biological samples/hr., alternatively at least about 300 biological samples/hr., alternatively at least about 400 biological samples/hr., alternatively at least about 440 biological samples/hr., or alternatively at least 500 biological samples/hr.

At operation, a biological sample and a reagent comprising capture antibody configured to bind to total ApoE are dispensed into a reaction vessel and mixed. The mixing can be performed with a stirrer in direct contact with the fluidic substances, an ultrasonic probe in direct or indirect contact with the fluidic substances, or any other suitable mixing apparatus. In some aspects, the immunoassay analyzer includes an ultrasonic mixing module. For instance, a reagent pipettor may be outfitted with a tip that allows it to perform ultrasonic mixing of a reagent in a reagent pack before aspirating it for transport to a reaction vessel, thereby ensuring that the aspirated reagent would not be impacted by any settling that may have taken place in the reagent pack. Sample pipettors may similarly be specialized.

The mixture is then transferred to an incubator. The transfer unit transfers the reaction vessels to and from the incubator station which includes an incubator. In some embodiments, the transfer unit transfers one or more of the pipetted reaction vessels from the reagent carriage unit to the incubator. Further, the transfer unit can transfer one or more reaction vessels from the incubator to the reagent carriage unit. The transfer unit can also remove from the reaction vessels that have been read or completed the incubator. The incubator is thermally controlled to maintain a predetermined temperature. In some embodiments, the incubator is maintained about 30 °C to 40 °C. In other embodiments, the incubator is maintained about 37 °C to ensure immunological reaction and enzyme reaction, for example. By way of example, the incubator performs assay incubation.

During the incubation, the biological sample and the capture antibody interact. The resulting “first reaction mixture” is a result of the incubation between the sample and the reagent.

In some embodiments, the transfer unit transfers incubated reaction vessels from the incubator to the wash unit, transfers assay reaction vessels from the wash unit to the incubator, transfers reaction vessels containing substrate from the wash unit to the incubator for substrate incubation or enzyme reaction, transfers washed reaction vessels from the incubator to detector arrangement after substrate incubation, and transfers the reaction vessels that have been read or completed from the detector arrangement to the incubator. The used reaction vessels can be delivered to a waste location.

At operation, a first portion of the first reaction mixture and a reagent comprising a first enzyme-conjugated affinity molecule configured a single isoform of ApoE at a different binding site than the capture antibody are dispensed into a reaction vessel and mixed. The mixture is then transferred to the incubator. During the incubation, the sample and the first enzyme-conjugated affinity molecule interact. The resulting “second reaction mixture” is a result of the incubation between the first enzyme-conjugated affinity molecule and the reagent.

At operation, a second portion of the first reaction mixture and a reagent comprising a second enzyme-conjugated affinity molecule configured to bind total ApoE at a different binding site than the capture antibody are dispensed into a reaction vessel and mixed. The mixture is then transferred to the incubator. During the incubation, the sample and the first enzyme-conjugated affinity molecule interact. The resulting “third reaction mixture” is a result of the incubation between the second enzyme-conjugated affinity molecule and the reagent.

In a non-limiting example, the incubation time of the first, second, and/or third reaction mixtures is at least about 30 minutes, alternatively at least about 40 minutes, alternatively at least about 50 minutes, alternatively at least about 55 minutes, or alternatively at least about 60 minutes.

The wash station receives and supports reaction vessels thereon such that various aspects of diagnostic process are performed with the automated analyzer. In an embodiment, the wash station is configured to wash away at least some of the unreacted components. Unreacted components may include unreacted reagents (e.g., free antigens, antibodies, unbound reactants, particles, and/or fluid, etc.) and unreacted sample. The wash station may be configured to perform a set number of wash actions depending on the assay. The wash station may also be configured to perform a set number of washes within a predetermined sequence. In certain embodiments, the wash station is configured to perform at least one wash action to wash away at least a portion of unreacted components, alternatively configured to perform at least two wash actions, alternatively configured to perform at least three wash actions, alternatively configured to perform at least four wash actions, alternatively configured to perform at least five wash actions, alternatively configured to perform at least six wash actions, alternatively configured to perform at least seven wash actions, alternatively configured to perform at least eight wash actions, alternatively configured to perform at least nine wash actions, or alternatively configured to perform at least ten wash actions. In some embodiments, the wash station is a thermally controlled device to separate bound or free analytes from particles after incubation. In some embodiments, the wash unit is maintained about 30 °C to 40 °C. In other embodiments, the wash unit is maintained about 37 °C to ensure enzyme reaction, for example, U.S. Patent Publication No. 2022/0357352, which is incorporated by reference in its entirety herein, discloses configurable wash processes according to an aspect of the invention.

The wash station may comprise a washing arrangement which may be configured to provide a base number of wash series (or wash actions) for each reaction vessel and optionally provide an additional number(s) of wash actions. The additional number(s) of wash actions may include one, a plurality, or all of a potential number of wash actions. An additional number of wash actions beyond the base number of wash actions may be specified for certain assays in an assay protocol file. The washing arrangement may include cleaning dispense nozzle (or probe) which dispenses a rinsing fluid and a cleaning aspiration nozzle (or probe) which aspirates the unreacted components. At operation, a base number of wash actions performed may be one, two, three, four, or five and the additional number of wash action(s) may be one, two, three, four or five. At operation, a base number of wash actions performed may be three and the additional number of wash action(s) may be one or two. In this embodiment, the base number of wash actions may be performed if three probes dispense buffer solution once per vessel and the three probes aspirate the at least some of the unreacted components some of the buffer solution, and/or the at least some of the unreacted reagents once per vessel. According to the principles of the present disclosure, certain probe(s) may be selectively used to dispense clean buffer solution into the vessel and aspirate the at least some of the unreacted components of the sample, some of the buffer solution, and/or the at least some of the unreacted reagents from the vessel to perform the additional wash action(s).

In an embodiment, the capture antibody is conjugated to at least one magnetic bead and the first reaction mixture is subjected to a magnetic field. The magnetic beads do not exhibit bead-to-bead attraction, only migrating when a magnetic field is applies. Captured analytes or targets are separated from the mixture and magnetization may be used to retain desired components within a reaction vessel.

At operation, the vessel containing the first reaction mixture is moved near one or more magnets. The one or more magnets attract the magnetic bead(s) or magnetic particle(s) to one or more sides of the reaction vessel. The reaction vessel is then subject to a wash process in which a cleaning dispense nozzle dispenses a rinsing fluid and a cleaning aspiration nozzle aspirates the unreacted components. The aspiration nozzle may be washed with a probe washer before and/or after the aspirating. The reaction vessel may undergo a series of wash process which may include at least two series of dispensing the rinsing fluid and aspirating the uncollected fluid components, alternatively at least three series, alternatively at least four series, alternatively at least five series. As a result, an unreacted substance or substances in the vessel is removed (e.g., rinsed away) by the bound-free cleaning aspiration nozzle.

At operation, a substrate is dispensed into the second reaction mixture. After mixing and a certain reaction time necessary for the substrate and the second reaction mixture to interact. The reaction vessel is then subject to a wash process in which a cleaning dispense nozzle dispenses a rinsing fluid and a cleaning aspiration nozzle aspirates the unreacted components. The aspiration nozzle may be washed with a probe washer before and/or after the aspirating. The reaction vessel may undergo a series of wash process which may include at least two series of dispensing the rinsing fluid and aspirating the uncollected fluid components, alternatively at least three series, alternatively at least four series, alternatively at least five series. As a result, an unreacted substance or substances in the vessel is removed (e.g., rinsed away) by the bound-free cleaning aspiration nozzle. The resulting detection mixture is transferred to the detector arrangement.

At operation, a substrate is dispensed into the third reaction mixture. After mixing and a certain reaction time necessary for the substrate and the second reaction mixture to interact. The reaction vessel is then subject to a wash process in which a cleaning dispense nozzle dispenses a rinsing fluid and a cleaning aspiration nozzle aspirates the unreacted components. The aspiration nozzle may be washed with a probe washer before and/or after the aspirating. The reaction vessel may undergo a series of wash process which may include at least two series of dispensing the rinsing fluid and aspirating the uncollected fluid components, alternatively at least three series, alternatively at least four series, alternatively at least five series. As a result, an unreacted substance or substances in the vessel is removed (e.g., rinsed away) by the bound-free cleaning aspiration nozzle. The resulting detection mixture is transferred to the detector arrangement.

Assays including features and/or characteristics described herein may benefit from one or more additional number(s) of wash actions.

In certain embodiments, the transport device includes three pick-and-place grippers, where a first pick-and-place gripper may be used to transport sample containers among the onload section, the transfer station, and reagent pipetting stations. A second pick-and-place gripper may be used to transport reaction vessels between the reagent pipetting stations and the incubator station or read station. A third pick-and-place gripper may be used to transport reaction vessels between the incubator station and the wash station or read station. A detailed description of the configurations and functions of one embodiment of the vessel pick-and-place grippers is provided in U.S. Pat. No. 7,128,874 and is incorporated herein in its entirety by reference. However, it should be understood that other pick-and-place mechanism that are capable of transporting sample and reaction vessels among the various modules of the automated analyzer is also contemplated for the purpose of the present invention.

In an aspect, the automated analyzer includes a machine vision apparatus comprising an image capture device and an image interpretation device configured to monitor instrument and/or assay functionalities of the automated analyzer. In some embodiments, instrument functionalities may include optical sensors, pressure sensors and thermistors. In some embodiments, the assay functionalities may include sample volume monitoring, total reagent volume monitoring, residual volume monitoring, and particle retention monitoring. The machine vision apparatus operates to evaluate the preparation of samples for subsequent analysis. In some embodiments, the machine vision apparatus utilizes one or more image capture units to determine whether samples have been appropriately prepared for analysis. As described herein, the machine vision apparatus provides direct and simple measurements of volume or integrity of a sample to determine whether the sample is appropriately prepared so that the analytic unit produces a reliable result. An exemplary machine vision apparatus is described in U.S. Pat. No. 11,263,433, which is incorporated by reference herein.

In some aspects, the machine vision apparatus operates to detect a volume of a fluidic substance in a container and determine whether the volume held in the container is appropriate as targeted. As described herein, this volume detection is configured to detect a volume at a dispense tip using the dispense tip image capture unit, and a volume at a vessel using a vessel image capture unit.

In some aspects, the machine vision apparatus operates to detect any interferents, which can interfere with an analytic procedure and may generate incorrect results in the dispense tip. As described herein, this dispense tip evaluation is configured to determine a quality of a fluidic substance at a dispense tip using a dispense tip image capture unit, and an alignment of the dispense tip with respect to the dispense tip image capture unit.

In some aspects, the machine vision apparatus operates to determine a particle concentration in a fluidic substance contained in a vessel, such as a reaction vessel, a sample vessel, a dilution vessel, a cuvette, or any suitable type of vessel, which is used throughout the process in the immunoassay analyzer. In some embodiments, this reaction vessel particle concentration check uses the vessel image capture unit.

The dispense tip image capture unit operates to capture images of dispense tips in one or more locations. In some embodiments, the dispense tip image capture unit is fixed at a particular location in the instrument. In other embodiments, the dispense tip image capture unit is movably disposed in the instrument, which can move either independently from other components of the instrument or together with one or more components of the instrument. Some embodiments of the instrument include a plurality of dispense tip image capture units. As described herein, the dispense tip image capture unit can include a camera unit.

The vessel image capture unit operates to capture images of vessels in one or more locations. In some embodiments, the vessel image capture unit is fixed at a particular location in the immunoassay analyzer. In other embodiments, the vessel image capture unit is movably disposed in the immunoassay analyzer, which can move either independently from other components of the immunoassay analyzer or together with one or more components of the immunoassay analyzer. Some embodiments of the immunoassay analyzer include a plurality of vessel image capture units. As described herein, the vessel tip image capture unit includes a camera unit.

The carriage image capture unit operates to capture images of container carriage devices with or without containers in one or more locations. In some embodiments, the carriage image capture unit is fixed at a particular location in the instrument. In other embodiments, the carriage image capture unit is movably disposed in the instrument, which can move either independently from other components of the instrument or together with one or more components of the instrument. Some embodiments of the instrument include a plurality of carriage image capture units.

All of the units of the automated analyzer are connected to a controller, which can perform block control of all of the analyzer functions by using, for example, a microcomputer. The controller may contain subunits such as a data processing unit, a communication interface, and others. A controller in accordance with an exemplary embodiment of the present technology may comprise a data processor, a non-transitory computer-readable medium, and a data storage coupled to the data processor. The non-transitory computer-readable medium may comprise code, executable by the data processor, to perform the functions described herein. The data processor may store, for example, data for processing samples, sample data, or data for analyzing sample data. Further, the non-transitory computer-readable medium may comprise code, executable by the data processor, to perform one or more of the method or process steps described herein.

The data processor may include any suitable data computation device or combination of such devices. An exemplary data processor may comprise one or more microprocessors working together to accomplish a desired function. The data processor may include a CPU that comprises at least one high-speed data processor adequate to execute program components for executing user and/or system-generated requests. The CPU may be a microprocessor such as AMD's Athlon, Duron and/or Opteron; IBM and/or Motorola's PowerPC; IBM's and Sony's Cell processor; Intel's Celeron, Itanium, Pentium, Xeon, and/or XScale; Apple M1, and/or the like processor(s).

The computer-readable medium and the data storage may be any suitable device or devices that can store electronic data. Examples of memories may comprise, for example, one or more memory chips, disk drives, etc. Such memories may operate using any suitable electrical, optical, and/or magnetic mode of operation.

The computer-readable medium may comprise code, executable by the data processor to perform any suitable method. For example, the computer-readable medium may comprise code, executable by the processor, to cause the controller to operate on a pre-determined schedule. In some embodiments of the presently claimed technology, the pre-determined schedule is a constituent test.

The presently described technology and its advantages will be better understood by reference to the following examples. These examples are provided to describe specific implementations of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### EXAMPLES

### Example 1: Exemplary ApoE4/Total ApoE Combination Assay

The assay is performed using an exemplary immunoassay analyzer having (i) four reagent pipettors and one sample pipettor; (ii) reagent packs configured to store volumes of reagents for at least 50 instances of each assay; (iii) an ultrasonic mixer; (iv) the capability to perform over 5 wash cycles per reaction vessel; and (v) a luminometer.

The reagent pack includes 5 reagent vessels. The first reagent vessel includes capture antibodies capable of binding to all major of isoforms of ApoE (“total ApoE”) conjugated to paramagnetic particles, the second reagent vessel includes ApoE4 monoclonal antibodies conjugated to alkaline phosphatase (“ALP”), the third reagent vessel includes an assay buffer, and the fourth reagent vessel includes ApoE antibodies capable of binding to all major isoforms of ApoE (“PAN-ApoE”) antibodies conjugated to ALP.

The paramagnetic particle conjugated with capture antibodies from the first reagent vessel (total ApoE capture antibodies) were pipetted into a reaction vessel using one of the four reagent pipettors along with a buffer. A first sample aliquot was pipetted into the reaction vessel using a sample pipettor. Then the reaction vessel was mixed using the ultrasonic mixer and incubated. A magnetic field was applied to the reaction vessel and the first incubated mixture was washed using a wash buffer to remove any unreacted components.

A secondary antibody capable of binding ApoE4 conjugated to ALP was added to the reaction vessel containing the incubated mixture. The reaction vessel was mixed ultrasonically and incubated, generating a first reaction mixture. A magnetic field was applied to the reaction vessel and the second reaction mixture was washed five times using a wash buffer to remove any unreacted components. A substrate was added to the reaction vessel, allowed to incubate, and the signal generated from the resulting reaction was read using a luminometer.

The paramagnetic particle conjugated with capture antibodies from the first reagent vessel (total ApoE capture antibodies) were pipetted into a reaction vessel using one of the four reagent pipettors along with a buffer. A second sample aliquot was pipetted into the reaction vessel using a sample pipettor. Then the reaction vessel was mixed using the ultrasonic mixer and incubated, generating a second incubated mixture. A magnetic field was applied to the reaction vessel and the second incubated mixture was washed using a wash buffer to remove any unreacted components.

A secondary PAN-ApoE antibody conjugated to ALP was added to the reaction vessel containing the second mixture. The reaction vessel was mixed ultrasonically and incubated, generating a second reaction mixture. A magnetic field was applied to the reaction vessel and the second reaction mixture was washed five times using a wash buffer to remove any unreacted components. A substrate was added to the reaction vessel, allowed to incubate, and the signal generated from the resulting reaction was read using a luminometer.

Test results were determined automatically by the system software. The signal generated by the assay was measured in relative light units (RLUs). The resulting signals were compared to generate an ApoE4 comparison value. The comparison value was then compared to reference ranges associated with various stages of AD. The subject is then assigned a degree of likelihood of developing AD based on the reference ranges. The subject was also assigned a zygosity for the APOE ε4 allele based on the comparison value.

### Example 2: ApoE4/total ApoE Combination Assay Native Patient Testing

An exemplary ApoE4/total ApoE combination assay was assessed for the ability to predict APOE4⁺⁺ (ApoE4 homozygous) populations, APOE4^{-/+} (ApoE4 heterozygous) populations, and APOE4^{-/-} populations.

The ApoE4/total ApoE combination assay was used to screen a panel of native patient samples on Beckman Coulter's DxI 9000 analyzer having (i) four reagent pipettors and one sample pipettor configured to aspirate and dispense less than about 10 μL; (ii) a reagent pack with 5 reagent vessels (a first reagent vessel containing total ApoE capture antibodies conjugated to paramagnetic particle, a second reagent vessel containing ApoE4 monoclonal antibodies conjugated to ALP, a third reagent vessel containing an assay buffer and a fourth reagent vessel containing PAN-ApoE monoclonal antibodies conjugated to ALP) where each reagent vessel is configured to store volumes of reagents for at least 50 instances of assays; (iii) an ultrasonic mixer; and (iv) the capability to perform over 5 wash cycles per reaction vessel.

Step 1: The paramagnetic particle conjugated with capture antibodies from the first reagent vessel (total ApoE capture antibodies) were pipetted into a reaction vessel using one of the four reagent pipettors along with a buffer. A first sample aliquot was pipetted into the reaction vessel using a sample pipettor. Then the reaction vessel was mixed using the ultrasonic mixer and incubated. A magnetic field was applied to the reaction vessel and the first incubated mixture was washed using a wash buffer to remove any unreacted components.

Step 2: A secondary antibody capable of binding ApoE4 conjugated to ALP was added to the reaction vessel containing the incubated mixture. The reaction vessel was mixed ultrasonically and incubated, generating a first reaction mixture. A magnetic field was applied to the reaction vessel and the second reaction mixture was washed five times using a wash buffer to remove any unreacted components. A substrate was added to the reaction vessel, allowed to incubate, and the signal generated from the resulting reaction was read using a luminometer.

Step 3: The paramagnetic particle conjugated with capture antibodies from the first reagent vessel (total ApoE capture antibodies) were pipetted into a reaction vessel using one of the four reagent pipettors along with a buffer. A second sample aliquot was pipetted into the reaction vessel using a sample pipettor. Then the reaction vessel was mixed using the ultrasonic mixer and incubated, generating a second incubated mixture. A magnetic field was applied to the reaction vessel and the second incubated mixture was washed using a wash buffer to remove any unreacted components.

Step 4: A secondary PAN-ApoE antibody conjugated to ALP was added to the reaction vessel containing the second mixture. The reaction vessel was mixed ultrasonically and incubated, generating a second reaction mixture. A magnetic field was applied to the reaction vessel and the second reaction mixture was washed five times using a wash buffer to remove any unreacted components. A substrate was added to the reaction vessel, allowed to incubate, and the signal generated from the resulting reaction was read using a luminometer.

All samples were analyzed in duplicate.

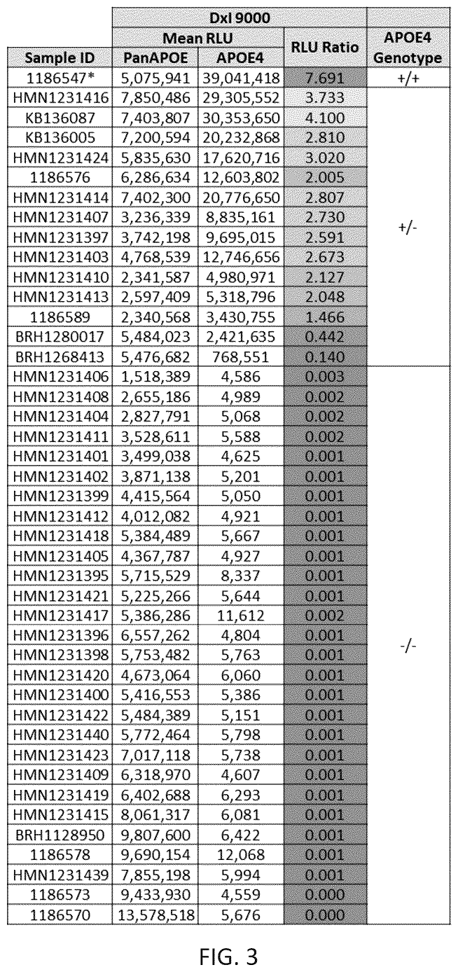

For each sample a ratio of the signal produced in Step 2 (ApoE4) to the signal produced in Step 4 (PanAPOE) was calculated. The calculated ratios were used to identify the sample as APOE4^{-/-}, APOE4^{-/+} (patients who are thought to have roughly a 3-fold increased risk of developing AD) or APOE4^{+/+} (patients who are thought to have roughly a 12-fold increased risk of AD). The results are depicted in FIG. 3.

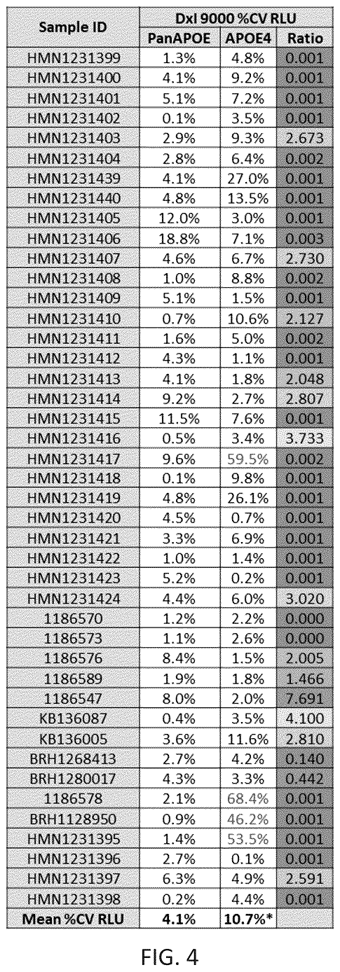

As shown in FIG. 4, % CV RLU was calculated for the signal produced in Step 2 (ApoE4) and the signal produced in Step 5 (PanAPOE) for the patient samples.

### Example 3: ApoE4/total ApoE Combination Assay Clinical Performance

An exemplary ApoE4/total ApoE combination assay was assessed for clinical performance including the ability to distinguish between APOE4^{+/+} (ApoE4 homozygous) populations, APOE4^{-/+} (ApoE4 heterozygous) populations, and APOE4^{-/-} populations. 298 samples were evaluated using the methods described in Example 2.

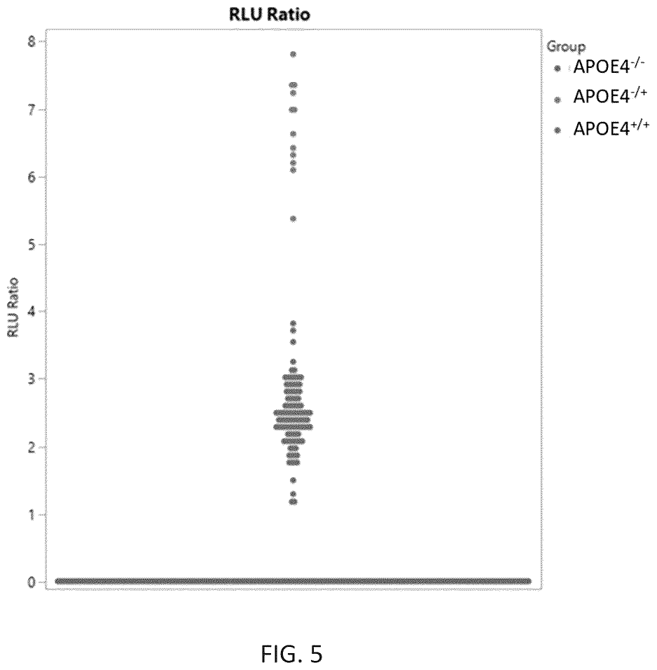

For each sample a ratio of the signal produced in Step 2 (ApoE4) to the signal produced in Step 4 (Total ApoE) was calculated. The calculated ratios were used to identify the sample as APOE4^{-/-}, APOE4^{-/+} (patients who are thought to have roughly a 3-fold increased risk of developing AD) or APOE4^{+/+} (patients who are thought to have roughly a 12-fold increased risk of AD). The results are depicted in FIG. 5.

Table 2 lists the minimum, maximum, mean and SD ratios for the patient samples categorized by the predicted genotype. The SD separation between the sample populations, APOE4^{+/+}/APOE4^{-/-} and APOE4^{-/+}/APOE4^{+/+}, is 5.2 for both, indicating that the patient samples for each genotype are well-separated and the difference between them is statistically significant.

TABLE 2
Results Summary for Patient Samples

Predicted Genotype

| | APOE4^{-/-} | APOE4^{-/+} | APOE4^{+/+} |
|---|---|---|---|
| Minimum Ratio | 0.003 | 5.372 | 1.156 |
| Max Ratio | 0.007 | 7.806 | 3.786 |
| Mean Ratio | 0.004 | 6.726 | 2.404 |
| SD Ratio | 0.001 | 0.684 | 0.462 |

The predicted genotype results were also compared with PCR results. As shown in Table 3, the ApoE4/total ApoE combination assay has >99% concordance to PCR genotype prediction. Concordance generally refers to the agreement or consistency between diagnostic tests or methods. The high concordance indicates that the ApoE4/total ApoE combination assay is a reliable and accurate tool for predicting APOE genotypes.

TABLE 3
Concordance Comparison

| | | ApoE4/total ApoE | |
|---|---|---|---|
| | PCR | Combination Assay | |
| | Genotype | Prediction | Concordance |
| APOE4^{-/-} | 181 | 181 | 100% |
| APOE4^{-/+} | 12 | 12 | 100.0% |
| APOE4^{+/+} | 107 | 105 | 98.1% |
| All | 300 | 298 | 99.3% |

### Example 4: Imprecision

An exemplary APOE ε4 assay was assessed using K2 EDTA plasma samples with variable APOE ε4 status (n=95). The samples were tested in duplicate on two exemplary immunoassay analyzers—the Beckman Coulter Access 2 immunoassay analyzer and the Beckman Coulter DxI 9000 immunoassay analyzer—using two reagent lots.

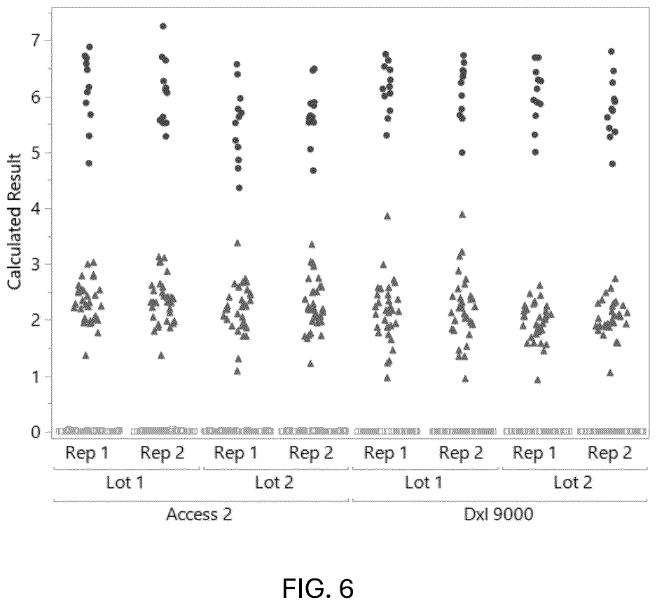

The frozen K2 EDTA plasma samples with variable APOE ε4 status were thawed by rocking at room temperature. After thawing, samples were centrifuged at 18,000×g for 10 minutes. PanAPOE and APOE ε4 relative light unit (RLU) values were then generated using the exemplary assay on both immunoassay platforms. Samples were tested with two reagent lots and two replicates per reagent lot on each platform. The ratio of APOE ε4 RLU/panAPOE RLU signals for each replicate, reagent lot, and immunoassay platform was then calculated and graphed using JMP 16 statistical modeling software. Low (square), medium (triangle) and high (circle) ratio values—corresponding to no ε4 allele, ε4 homozygous, and ε4 heterozygous subject statuses respectively—were identified based on the first rep of the first lot on the Access 2 platform. Per-rep calculated results were then graphed to highlight the low variability of the assay (FIG. 6). The exemplary immunoassay showed 100% agreement in results interpretation across reps, reagent lots and testing platform.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

It will be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A system for assessing a relative presence of a first analyte subgroup of a total analyte in a sample, wherein the total analyte present in the sample includes the first analyte subgroup and at least one additional analyte subgroup, the system comprising:
a. an immunoassay analyzer comprising a pipettor arrangement;
b. an analytic unit;
c. a detector arrangement; and
d. a non-transitory computer-readable medium storing instructions that, when executed by a processor, cause the system to perform a series of steps comprising:
i. a first step comprising assaying the sample to produce a first quantitative signal corresponding to the first analyte subgroup;
ii. a second step comprising assaying the sample to produce a second quantitative signal corresponding to the total analyte or the at least one additional analyte subgroup; and
iii. a third step comprising determining the relative presence of the first analyte subgroup of the total analyte in the sample by relatively comparing the two quantitative signals without reference to a calibration curve,
wherein either all subgroups of the total analyte in the sample share a common epitope, or the first analyte subgroup and the at least one additional analyte subgroup share a common epitope; and
wherein the assaying the sample steps to produce the first and second quantitative signals further comprise binding a capture agent to the common epitope.

2. The system of claim 1, wherein the first step of assaying the sample to produce the first quantitative signal and the second step of assaying the sample to produce the second quantitative signal are not performed concurrently.

3. (canceled)

4. The system of claim 1, wherein the first step of assaying the sample to produce the first quantitative signal further comprises exposing the sample to a first detection agent specific to the first analyte subgroup in the sample and the second step of assaying the sample to produce the second quantitative signal corresponds to the total analyte present in the sample and further comprises exposing the sample to a second detection agent capable of binding to the total analyte in the sample.

5. The system of claim 1, wherein the first step of assaying the sample to produce the first quantitative signal further comprises exposing the sample to a first detection agent specific to the first analyte subgroup in the sample and the second step of assaying the sample to produce the second quantitative signal further comprises exposing the sample to a second detection agent specific to the at least one additional analyte subgroup in the sample.

6. The system of claim 1, wherein the first and second quantitative signals are generated based on bulk signal accumulation and not individual binding events.

7. The system of claim 1, wherein the first and second steps comprising assaying the sample comprise the use of magnetic particle-based immunoassays performed in two separate reaction vessels.

8. The system of claim 7, wherein the immunoassays performed are subject to differing reaction conditions that may comprise different incubation times, mix times, or reagent additions.

9. (canceled)

10. The system of claim 1, wherein the analyte comprises a peptide, a polypeptide, an enzyme, a protein, a protein complex, a monoclonal antibody, a polyclonal antibody, an antibody fragment, a hormone, a synthetic antibody mimic, an aptamer, an affimer, DARPins, oligonucleotide, a nucleic acid, or an antigen.

11. (canceled)

12. The system of claim 1, wherein the sample comprises a biological sample from a patient, wherein the biological sample is serum, whole blood, plasma, cerebral spinal fluid, urine, feces, sputum, tissue, marrow, semen, mucus, or nasopharyngeal fluid.

13. (canceled)

14. The system of claim 7, wherein the immunoassay comprises a chemiluminiscence immunoassay (CLIA), immunofluorescence assay, fluorescence polarization assay, one-site noncompetitive assay, or two-site noncompetitive (sandwich) immunoassay.

15. (canceled)

16. The system of claim 1, wherein: the first step comprising assaying the sample to produce a first quantitative signal corresponding to the first analyte subgroup comprises:
i. contacting a first portion the sample with a capture antibody specific for the analyte to produce a first capture mixture;
ii. contacting the first capture mixture with a detector affinity molecule that is specific for the first analyte subgroup to produce a first detection mixture;
iii. conducting a first detection reaction in the first detection mixture generating a first quantitative signal; and
iv. detecting the first quantitative signal; and
the second step comprising assaying the sample to produce a second quantitative signal corresponding to the total presence of analyte or the at least one additional analyte subgroup comprises:
i. contacting a second portion the sample with a capture antibody specific for the analyte to produce a second capture mixture;
ii. contacting the second capture mixture with a detector affinity molecule that is specific for the analyte or the at least one additional analyte to produce a second detection mixture;
iii. conducting a second detection reaction in the second detection mixture generating a second quantitative signal; and
iv. detecting the second quantitative signal.

17. The system of claim 16, wherein the detector affinity molecule is an antibody, a monoclonal antibody, a polyclonal antibody, an antibody fragment, a synthetic antibody mimic, an aptamer, an affimer, DARPins, oligonucleotide, peptide, or antigen.

18. The system of claim 16, wherein the first capture antibody and/or the second capture antibody is coupled to a particle or bead that is optionally magnetic.

19. The system of claim 16, wherein the first detector affinity molecule, and/or the second detector affinity molecule is conjugated to at least one enzyme.

20. (canceled)

21. (canceled)

22. (canceled)

23. The system of claim 1, wherein the two quantitative signals are generated via a detection reaction between the first detector affinity molecule and/or the second detector affinity molecule and a substrate formulation, wherein the substrate formulation comprises:
a chemiluminescent compound of the formula A or a salt thereof: 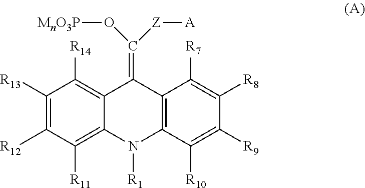
wherein
A is C₁₋₆haloalkyl, naphthyl, phenyl, substituted phenyl, or heteroaryl, wherein substituted phenyl comprises from 1 to 3 halo, C₁₋₆ alkyl, C₁₋₆ alkoxy, C₁₋₆ haloalkyl, C(O)R₁₅, CN or NO₂ substituents;
R₁ is selected from the group consisting of C₅₋₁₄aryl, C₁₋₆ alkyl, C₁₋₆ haloalkyl, and C₅₋₁₄ aralkyl groups;
R₇-R₁₄ are independently H, C₁₋₆ alkoxy, halo, C₁₋₄ alkyl, or R₇ or R₈-R₉ or R₉-R₁₀ or R₁₁-R₁₂ or R₁₂-R₁₃ or R₁₃-R₁₄, can be joined together as a carbocyclic or heterocyclic ring system comprising at least one 5 or 6-membered ring;
R₁₅ is C₁₋₆ alkyl;
each M is independently selected from the group consisting of H, an alkali metal, alkaline earth metal, transition metal, ammonium, phosphonium, organic amine salt, and an amino acid salt;
Z is O or S; and
n is 0, 1, or 2;
a cationic aromatic compound (CAC);
a background reducing agent; and
an ether-linked nonionic surfactant or a hydrophilic polymer.

24. The system of claim 1, wherein the immunoassay analyzer further comprises at least one reagent pack configured to hold a plurality of reagent vessels, wherein the reagent pack comprises a reagent vessel comprising the first and second capture antibodies, a reagent vessel comprising the first detector affinity molecule, and a reagent vessel comprising the second detector affinity molecule.

25. (canceled)

26. (canceled)

27. The system of claim 1, wherein the immunoassay analyzer further comprises: a machine vision apparatus comprising an image capture device and an image interpretation device configured to monitor instrument and, optionally, one or more assay instrument functionalities selected from the group consisting of optical sensors, pressure sensors thermistors, sample volume monitoring, total reagent volume monitoring, residual volume monitoring, particle retention monitoring, and an ultrasonic mixing module.

28. (canceled)

29. (canceled)

30. The system claim 1, wherein the pipettor arrangement comprises at least a first reagent pipettor, a second reagent pipettor, and a third reagent pipettor.

31. (canceled)

32. (canceled)

33. The system claim 1, wherein:
a. the first step comprising assaying the sample is done in a first reaction vessel to produce a first reaction mixture comprising unreacted components, and the immunoassay analyzer further comprises a washing arrangement, wherein the washing arrangement is configured to perform at least one wash action to wash away at least a portion of unreacted components; and
b. the second step comprising assaying the sample is done in a second reaction vessel to produce a second reaction mixture comprising unreacted components, and the immunoassay analyzer further comprises a washing arrangement, wherein the washing arrangement is configured to perform at least one wash action to wash away at least a portion of unreacted components.

34. The system of claim 33, wherein the first reaction mixture and/or the second reaction mixture is subjected to a magnetic field prior to performing the at least one wash action.

35-94. (canceled)


SEQUENCE LISTING (3 sequences)

SEQ ID NO: 1 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKCLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 2 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVCGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH

SEQ ID NO: 3 - protein, 299 aa, Homo sapiens
KVEQAVETEPEPELRQQTEWQSGQRWELALGRFWDYLRWVQTLSEQVQEELLSSQVTQELRALMDETMKELKAYKSELEEQLTPVAEETRARLSKELQAAQARLGADMEDVRGRLVQYRGEVQAMLGQSTEELRVRLASHLRKLRKRLLRDADDLQKRLAVYQAGAREGAERGLSAIRERLGPLVEQGRVRAATVGSLAGQPLQERAQAWGERLRARMEEMGSRTRDRLDEVKEQVAEVRAKLEEQAQQIRLQAEAFQARLKSWFEPLVEDMQRQWAGLVEKVQAAVGTSAAPVPSDNH